(19) United States
(12) Patent Application Publication
(10) Pub. No.: US 2004/0003432 A1
(43) Pub. Date: Jan. 1, 2004

(54) Production of hexosamines and uses thereof

(75) Inventor: Mark G. Obukowicz, Kirkwood, MO (US)

Correspondence Address: Robert S. Thomas, Keenan Building, Third Floor, 1330 Lady Street, Columbia, SC, 29201, US

(73) Assignee: Pharmacia Corporation, St. Louis, MO

(21) Appl. No.: 10/429,812

(22) Filed: May 5, 2003

Related U.S. Application Data
(60) Provisional application No. 60/378,297, filed on May 6, 2002.

(51) Int. Cl. A01H 1/00; C12N 15/82; C12P 19/28; C07H 5/04; C12N 5/04

(52) U.S. Cl. 800/284; 435/85; 536/18.7; 435/419

(57) ABSTRACT

A method of producing a hexosamine comprises providing a cell having genes encoding each enzyme required for a biosynthetic pathway capable of synthesizing the hexosamine where at least one gene in the pathway is a heterologous gene. Compositions and methods of producing transgenic cells, expression vectors, transgenic plants, and nutritional material that contain hexosamines are also provided, as are methods for preventing, treating, and inhibiting arthritis and articular-joint damage or disease in subjects in need of such prevention, treatment and/or inhibition.

### CROSS-REFERENCE TO RELATED PATENTS AND PATENT APPLICATIONS

This application claims priority to U.S. Provisional Application No. 60/378,297, filed May 6, 2002, which application is incorporated by reference herein in its entirety.

### BACKGROUND OF THE INVENTION

1. Field of the Invention

The present invention relates generally to compositions and methods for producing a hexosamine, and to the use of the hexosamine for prophylactic and/or therapeutic reasons, such as, for example, the prevention, treatment, or inhibition of pain, inflammation, or inflammation-related disorders.

2. Description of Related Art

Osteoarthritis and rheumatoid arthritis are diseases that affect joint cartilage. Osteoarthritis has an inflammation component, but also it is believed to be involved with the normal wear and tear on the protective cartilage that lines ends of the bones within the joints. In contrast, rheumatoid arthritis is thought to be an autoimmune disease in which the body's own cells attack the synovial membranes that cover, lubricate, and nourish the cartilage lining the joints at the end of bones.

Symptoms for both diseases usually include inflammation, pain, stiffness, crackling, and swelling of the affected joints. Eventually, both of these diseases can result in the painful degradation and deformation of the articular cartilage and bone. Rheumatoid arthritis can sometimes spread inflammation to other areas of the body and is typically the more severe of the two diseases.

Historically, physicians have treated the signs and symptoms of inflammation with a regimen of nonsteroidal anti-inflammatory drugs (NSAIDS), such as, for example, aspirin and ibuprofen. Undesirably, however, some NSAIDS are known to cause gastrointestinal (GI) bleeding or ulcers in patients undergoing consistent long term regimens of NSAID therapy. In some instances, selective cyclooxygenase-2 inhibitors, e.g. celecoxib, are supplanting non-selective NSAID's.

Recently, investigators have reported that glucosamine may be useful for the treatment of arthritis. Glucosamine is an amino sugar found generally in mucopolysaccharides, cell membranes, and in chitin derived from crustaceans.

Glucosamine occurs naturally in vertebrates as a product of the hexosamine biosynthetic pathway. In this pathway, it is believed that L-glutamine:D-fructose-6-phosphate transaminase (which may also be referred to herein as glutamine:fructose-6-phosphate amidotransferase, or GFAT) is the rate limiting enzyme in the synthesis of glucosamine. GFAT uses glutamine as a nitrogen donor and diverts 2-5% of the body's fructose-6-phosphate to synthesize glucosamine-6-phosphate, which is later converted into glucosamine. See, e.g., Sayeski et al., Nucleic Acids Research, 25 (7):1458-1466 (1997). Several additional biosynthetic steps later convert glucosamine-6-phosphate into n-acetyl-glucosamine.

Although not known to directly inhibit the Cox-2 enzyme, naturally occurring hexosamine amino sugars, such as glucosamine and galactosamine, have been reported to decrease inflammation and promote cartilage regeneration in arthritis patients. Glucosamine and galactosamine are monosaccharide precursor molecules used by both chondrocytes and synoviocytes to form disaccharide units via β1-3, β1-4, or β1-6 linkages with other monosaccharides. Chondrocytes and synoviocytes utilize the hexosamines, galactosamine and glucosamine, as fundamental building blocks for the body's production of cartilage. It has been proposed that supplementation of these hexosamines to the body should stimulate additional cartilage formation. See Piperno et al., Osteoarthr Cartil 8:207-212 (2000).

Accordingly, the popular press has recently reported the joint-protective properties of glucosamine in the management of arthritis for humans and animals. Most recently, indications of long-term, positive effects of glucosamine on osteoarthritis progression in humans have been published. See Reginster et al., The Lancet, 357:251-256 (2001). This clinical trial showed that glucosamine had combined efficacy as a structure-modifying and symptom-modifying agent, and provided the first data to suggest that glucosamine supplementation can repair the damage caused by arthritis, which in turn can reduce associated inflammation and pain. Other recent publications show similar cartilage growth in joint spaces among patients receiving 1,500 mg of glucosamine daily, compared with the placebo, over a three year period. See Pavelk et al., Arthritis Rheum 43:S384 (2000).

Galactosamine has also been reported to repair the damage caused by arthritis inflammation and pain, instead of simply addressing the symptoms. See Bali J., et al., Semin Arthritis Rheum 3(1):58-68 (2001). Typically, galactosamine has been manufactured by an expensive and laborious process through the extraction of chondroitin sulfate from bovine, porcine, and shark cartilage. In addition to the expense and labor, isolating nutraceuticals from animal bone and cartilage carries some risk for infection and disease transmission to mammals who consume these materials.

Likewise, glucosamine has also been manufactured by a laborious and expensive extraction process, which usually involves acid hydrolysis of the chitin found in crustacean shells. For example, U.S. Pat. No. 5,998,173 to Haynes et al., teaches a method for producing n-acetyl-glucosamine by chitinase enzymatic hydrolyzation of the chitin found in crustacean shells. Once the glucosamine and galactosamine via chondroitin sulfate is extracted from animal cartilage or crustacean shells, both are commonly packaged into gelatin capsules as an oral nutraceutical or into glass vials for intramuscular injection.

It is believed that conventional methods for producing glucosamine and galactosamine involve elaborate extraction procedures and often produce inefficient yields that often differ in purity from batch to batch. Such existing methods are also constrained by the need for a steady supply of chitin from crustacean shells and cartilage from animals. Also, some patients, including animals, are adverse to swallowing medication in a pill form or receiving an injection from a needle.

Accordingly, it would be useful if other sources of hexosamine amino sugars could be found that provide an alternative to existing manufacturing methods and offered different forms for administration.

From the foregoing, it can be seen that it would be useful to provide an improved method of producing an amino sugar hexosamine. It would also be useful if such hexosamine could be produced without compromising its clinical effectiveness and could be provided in a form that would be acceptable for delivery to a broad class of patients. In some instances, it would be useful to provide methods for the administration of a hexosamine to subjects by routes that differ from capsules or injections.

### SUMMARY OF THE INVENTION

The present invention is directed to a novel method for producing a hexosamine comprising providing a cell comprising polynucleotide sequences which encode for enzymes required for a biosynthetic pathway capable of synthesizing the hexosamine, where at least one of the polynucleotide sequences comprises a recombinant polynucleotide.

The present invention is also directed to a novel method of producing a hexosamine comprising transforming a cell with at least one heterologous polynucleotide coding for a polypeptide in a biosynthetic pathway that is capable of producing a hexosamine; and culturing the transformed cell under conditions that permit the cell to translate the polynucleotide into a polypeptide comprising an enzyme which is part of the biosynthetic pathway.

The present invention is also directed to a novel method of producing a hexosamine by a living cell comprising constructing at least one heterologous vector encoding for at least one gene encoding a polypeptide that functions as an enzyme in a hexosamine biosynthetic pathway, wherein the gene is a polynucleotide operably linked to a regulatory promoter sequence; transforming at least one cell with the heterologous vector; culturing the cell under conditions which allow for expression of the polynucleotide into the polypeptide; and permitting the cell to produce the hexosamine.

The present invention is also directed to a novel method of producing hexosamines in transgenic plants comprising constructing at least one heterologous vector encoding for at least one gene in a hexosamine biosynthetic pathway and one gene for a plant selectable marker; linking operably the hexosamine pathway gene and the plant selectable marker gene to at least one regulatory promoter that controls expression of said genes, wherein the expression of the genes is capable of producing a polypeptide for enzymatic hexosamine production and a polypeptide for the selectable marker; transforming a plant cell with either one or both of the genes operably linked to the at least one regulatory promoter; culturing the transformed plant cell under conditions that allow for the plant cell to regenerate into a plant; and growing the transformed plant under conditions that allow for the plant to produce the hexosamine.

The present invention is also directed to a novel transgenic plant that is capable of producing a hexosamine, the plant comprising at least one heterologous vector encoding for at least one gene in a hexosamine biosynthetic pathway, wherein the gene is linked operably to at least one regulatory promoter that controls expression of the gene.

The present invention is also directed to a novel recombinant expression system capable of expressing in host cells either one or both of: a gene encoding for a glutamine:fructose-6-phosphate amidotransferase enzyme comprising a polynucleotide sequence that is substantially similar to SEQ ID NO: 1; and a gene encoding for a phosphatase enzyme comprising a polynucleotide sequence that is substantially similar to SEQ ID NO: 13, both genes being operably associated with a regulatory promoter sequence that controls expression of the genes, wherein the expression system is capable of producing glucosamine in the host cells.

The present invention is also directed to a novel edible material comprising a hexosamine produced from a recombinant source.

The present invention is also directed to a novel edible material comprising plant material containing a hexosamine, wherein the plant is a transgenic plant that has been engineered to produce hexosamines.

The present invention is also directed to a novel method of producing glucosamine from a transgenic plant, the method comprising transforming a cell of the plant with a nucleic acid molecule having a heterologous nucleotide sequence substantially similar to SEQ ID NO: 1 encoding a glutamine:fructose-6-phosphate amidotransferase enzyme and a nucleic acid molecule having a heterologous nucleotide sequence substantially similar to SEQ ID NO: 13 encoding a phosphatase enzyme, both nucleic acids being operably linked to a regulatory promoter element; culturing the transformed plant cell under conditions which allow the plant cell to regenerate into a plant; growing the plant under conditions which allow the heterologous nucleic acid molecules to express the glutamine:fructose-6-phosphate amidotransferase and glucosamine-6-phosphatase enzymes; allowing the heterologous enzyme glutamine:fructose-6-phosphate amidotransferase to convert fructose-6-phosphate into glucosamine-6-phosphate and the heterologous enzyme glucosamine-6-phosphatase to convert the glucosamine-6-phosphate into glucosamine; extracting the glucosamine from the plant; and purifying the extracted glucosamine into a pharmaceutically accepted form. If desirable, the glucosamine can be eaten directly in foodstuffs.

The present invention is also directed to a novel transgenic host cell that is capable of producing a hexosamine, the cell comprising a cell having genes encoding each enzyme required for a biosynthetic pathway capable of synthesizing the hexosamine where at least one gene in the pathway is a heterologous gene.

The present invention is also directed to a novel method for the prevention, treatment and/or inhibition of pain, inflammation, and/or an inflammation-related disorder in a subject in need of such prevention, treatment, and/or inhibition, the method comprising administering to the subject a hexosamine-containing material which comprises polynucleotide sequences which encode for enzymes required for a biosynthetic pathway capable of synthesizing the hexosamine, where at least one of the polynucleotide sequences comprises a recombinant polynucleotide.

The present invention is also directed to a novel method for the prevention, treatment and/or inhibition of pain, inflammation, and/or an inflammation-related disorder in a subject in need of such prevention, treatment, and/or inhibition, the method comprising administering to the subject a hexosamine that is a product of a biosynthetic pathway which comprises enzymes encoded for by polynucleotide sequences, where at least one of the polynucleotide sequences comprises a recombinant polynucleotide.

Among the several advantages found to be achieved by the present invention, therefore, may be noted the provision of an improved method of producing an amino sugar hexosamine, and also the provision of a method of producing the hexosamine without compromising its clinical effectiveness and in a form that is acceptable for delivery to a broad class of patients, and also the provision of methods for the administration of a hexosamine to subjects by routes that differ from capsules or injections.

### BRIEF DESCRIPTION OF THE DRAWINGS

FIG. 1 is a graph showing the percent inhibition of IL-1-beta-induced degradation of bovine nasal cartilage as a function of glucosamine concentration and indicating the efficacy of glucosamine as an inhibitor of IL-1 induced degradation of bovine nasal cartilage in a concentration dependent manner.

### DETAILED DESCRIPTION OF THE PREFERRED EMBODIMENTS

In accordance with the present invention, it has been discovered that hexosamine sugars, which may also be referred to herein as “hexosamines” or “hexosamino sugars”, such as glucosamine and galactosamine, can be produced in plant cells or other cells that cannot normally synthesize those compounds. In fact, it is believed that such hexosamines can be produced in growing plants, and, in particular, in selected parts of such plants. In the present invention, one or more genes that encode for the production of an enzyme required by a particular host cell for the synthesis of a desired hexosamine is obtained from a source organism and is used to transform the host cell in a manner that permits the gene(s) to be expressed at a level that enables the host cell to produce the hexosamine. In other words, the foreign gene(s) complete a functioning biosynthesis pathway in the host cell for production of the desired hexosamine. By selecting the correct genes, a host cell can be transformed so that it is capable of producing a particular hexosamine, or two or more different types of hexosamine. In preferred embodiments, the hexosamine that is produced accumulates in the cell to provide hexosamine in concentrations that are useful for direct application, or for further isolation and purification.

The present invention also provides methods for the administration of a hexosamine to a subject by routes that are an alternative to capsules or injections. For example, it is believed that the hexosamines can be produced in the edible portions of plants. Consumption of those portions of the plants as a nutritional agent can provide a supply of the hexosamine to the subject in a conventional and easily consumable form. By way of example, hexosamines in this form would be useful for the treatment of arthritis, particularly, osteoarthritis in humans and companion animals. Moreover, hexosamine-containing products in this form have the advantage that the therapeutic effectiveness of the hexosamine can be maintained and the hexosamine can be provided in a delivery format that is more acceptable to a broader class of subjects.

The present invention also includes a food or beverage comprising a transgenic plant, or a part, extract, or product thereof, that contains a polynucleotide of the invention. In other embodiments, hexosamine-containing products derived from the transgenic plant may be free of such polynucleotides. These transgenic plants, especially those transgenic plants which produce hexosamines by virtue of the presence of the polynucleotide or polynucleotides, may constitute the food itself or they may be processed to form the food or beverage. For example, a tuber of a transgenic potato of the invention constitutes a food of this aspect of the invention. Alternatively, such a transgenic potato may be processed into another foodstuff which is, nevertheless, a food of this aspect of the invention.

In addition, the present invention provides the production of hexosamines in transgenic organisms other than plant cells. Hexosamines that are produced by transgenic fungi—and from transgenic yeasts in particular, bacteria, or the like, can be isolated from the producing cell, or can be used along with the cell itself. Some types of these transgenic, hexosamine-synthesizing cells can be added back to edible foods or to nutraceutical formulations to enhance the hexosamine content of the food or the formulation.

Unless otherwise indicated, the following terms are intended to have the following meanings in interpreting the present invention.

As used herein, the term “cDNA” means complementary deoxyribonucleic acid.

As used herein, the term “coding” means a polynucleotide sequence that is translated in an organism to produce a protein. The term “coding DNA sequence” is also a DNA sequence from which the information for making a peptide molecule, mRNA or tRNA are transcribed. A DNA sequence may be a gene, combination of genes, or a gene fragment.

As used herein, “complementary polynucleotides” are polynucleotides that are capable of base-pairing according to the standard Watson-Crick complementarity rules. Specifically, purines will base pair with pyrimidines to form combinations of guanine paired with cytosine (G:C) and adenine paired with either thymine (A:T) in the case of DNA, or adenine paired with uracil (A:U) in the case of RNA. Two polynucleotides may hybridize to each other if they are complementary to each other, or if each has at least one region that is substantially complementary to the other.

As used herein, the term “DNA” means deoxyribonucleic acid.

As used herein, the terms “edible plant material” or “edible plant parts” include a plant or any material obtained from a plant which is suitable for ingestion by a mammal or other animal including a human. These terms are intended to include raw plant material that may be fed directly to animals, including humans, or any processed plant material that is fed to animals, including humans. Materials obtained from a plant are intended to include any component of a plant which is eventually ingested by a human or other animal. The terms “edible plant parts” are meant to include any plant material that can be directly ingested by animals or humans as a nutritional source or dietary complement.

As used herein, the term “gene” should be understood to refer to a unit of heredity. Each gene is composed of a linear chain of deoxyribonucleotides which can be referred to by the sequence of nucleotides forming the chain. Thus, “sequence” is used to indicate both the ordered listing of the nucleotides which form the chain, and the chain itself, which has that sequence of nucleotides. (“Sequence” is used in the similar way in referring to RNA chains, linear chains made of ribonucleotides). The gene may include regulatory and control sequences, sequences which can be transcribed into an RNA molecule, and may contain sequences with unknown function. The majority of the RNA transcription products are messenger RNAs (mRNAs), which include sequences which are translated into polypeptides and may include sequences which are not translated. It should be recognized that small differences in nucleotide sequence for the same gene can exist between different strains of the same type of organism, or even within a particular strain of the organism, without altering the identity of the gene.

As used herein, the term “heterologous” is used to indicate a recombinant DNA sequence in which the promoter or regulator DNA sequence and the associated DNA sequence are isolated from organisms of different species or genera.

As used herein, the term “hexosamine” is any six-carbon sugar molecule having at least one amino group. By way of example and not intended as a complete list, several known hexosamines are glucosamine, galactosamine, and mannosamine. Also included in the use of the term “hexosamine” is any similar biochemical species having a hexosamine at its core structure. For example, the hexosamine, glucosamine, can be used interchangeably and stay within the present invention with: glucosamine-6-phosphate, glucosamine-1-phosphate, n-acetyl-glucosamine-6-phosphate, n-acetyl-glucosamine-1-phosphate, and UDP-n-acetyl-glucosamine. Also, use of the term “hexosamine” within the present invention, also includes any pharmaceutically acceptable salts. For example, two salts that can be considered to lie within the meaning of hexosamine are glucosamine sulfate and glucosamine HCl. Other forms of hexosamines such as galactosamine, mannosamine, neuraminic acid and muramic acid, along with any variation on their core structure, are also considered to be used interchangeably with hexosamine in much the same way as the glucosamine examples.

As used herein, the terms “hexosamine biosynthesis/biosynthetic pathway” refer to any one or more gene(s) or polynucleotide(s) known to encode for a polypeptide(s) that is(are) responsible for or assists in the production of hexosamines. The terms “pathway” or “biosynthetic pathway” refer to a connected series of biochemical reactions normally occurring in a cell, or more broadly a cellular event such as cellular division or DNA replication. Typically, the steps in such a biochemical pathway act in a coordinated fashion to produce a specific product or products or to produce some other particular biochemical action. Such a biochemical pathway requires the expression product of a gene if the absence of that expression product either directly or indirectly prevents the completion of one or more steps in that pathway, thereby preventing or significantly reducing the production of one or more normal products or effects of that pathway.

A polynucleotide may be “introduced” or “transformed” into any cell by any means known to those of skill in the art, including transfection, transformation or transduction, transposable elements, electroporation, Agrobacterium infection, polyethylene glycol-mediated uptake, particle bombardment, and the like. The introduced polynucleotide may be maintained in the cell stably if it is incorporated into a non-chromosomal autonomous replicon or integrated into the genome of the host cell. Alternatively, the introduced polynucleotide may be present on an extra-chromosomal non-replicating vector and be transiently expressed or transiently active. As used herein, the term “transform” refers to the introduction of a polynucleotide (single or double stranded DNA, RNA, or a combination thereof) into a living cell by any means. Transformed cells, tissues and plants encompass not only the end product of a transformation process, but also the progeny thereof, which retain the polynucleotide of interest.

As used herein, the term “isolated polynucleotide” is a polynucleotide that is substantially free of the nucleic acid sequences that normally flank the polynucleotide in its naturally occurring replicon. For example, a cloned polynucleotide is considered isolated. Alternatively, a polynucleotide is considered isolated if it has been altered by human intervention, or placed in a locus or location that is not its natural site, or if it is introduced into a cell by agroinfection.

As used herein, a “normal” form of a gene (wild type) is a form commonly found in natural populations of an organism. Commonly a single form of a gene will predominate in natural populations. In general, such a gene is suitable as a normal form of a gene, however, other forms which provide similar functional characteristics may also be used as a normal gene. In particular, a normal form of a gene does not confer a growth conditional phenotype on the organism having that gene, while a mutant form of a gene suitable for use in these methods can provide such a growth conditional phenotype.

As used herein, “nucleic acid” and “polynucleotide” refer to RNA or DNA that is linear or branched, single or double stranded, or a hybrid thereof. The polynucleotides of the invention may be produced by any means, including genomic preparations, cDNA preparations, in vitro synthesis, RT-PCR and in vitro or in vivo transcription. The term “polynucleotide” means a chain of at least two nucleotides joined together. The chain may be linear, branched, circular or combinations thereof. Nucleotides are generally those molecules selected for base-pairing and include such molecules as guanine, cytosine, adenine, uracil, and thymine.

As used herein, the term “operatively linked to” or “associated with” means two DNA sequences which are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

The “percent sequence identity” between two polynucleotide or two polypeptide sequences is determined according to either the BLAST program (Basic Local Alignment Search Tool; Altschul and Gish, Meth Enzymol 266:460-480 (1996); and Altschul, J. Biol 215:403-410 (1991), in the Wisconsin Genetics Software Package (Devererreux et al., Nucl Acid Res 12:387 (1984)), Genetics Computer Group (GCG) program, Madison, Wis. (NCBI, Version 2.0.11, default settings) or using Smith Waterman Alignment program (Smith and Waterman, Adv Appl Math 2:482 (1981)) as incorporated into GeneMatcher Plus™ (Paracel, Inc., using the default settings and the version current at the time of filing).

As used herein, two sequences are “substantially similar” when the percent sequence identity between the two sequences is at least 70%. It is preferred that substantially similar sequences have a percent sequence identity of at least 75%, more preferred of at least 80%, even more preferred of at least 85%, and yet more preferred of at least 90%, or even higher.

As used herein, the term “plant” refers to whole plants, plant organs and tissues (e.g., stems, roots, ovules, fruit, stamens, leaves, embryos, meristematic regions, callus tissue, gametophytes, sporophytes, pollen, microspores and the like), seeds, plant cells and the progeny thereof.

As used herein, the term “plant tissue” is any tissue of a plant in its native state or in culture. This term includes, without limitation, whole plants, plant cells, plant organs, plant seeds, protoplasts, callus, cell cultures, and any group of plant cells organized into structural and/or functional units. The use of this term in conjunction with, or in the absence of, any specific type of plant tissue as listed above or otherwise embraced by this definition is not intended to be exclusive of any other type of plant tissue.

Plants suitable for transformation according to the processes of this invention include, without limitation, monocots, such as corn, wheat, barley, sorghum, rye, rice, banana, and plantains, and dicots, such as potato, tomato, alfalfa, soybean, beans in general, canola, apple, pears, fruits in general, and other vegetables. Also included are plants such as cotton, flax, fiber-producing plants in general, trees, shrubs, and the like.

As used herein, the term “plant transformation vector” is a plasmid or viral vector that is capable of transforming plant tissue such that the plant tissue contains and expresses DNA that was not pre-existing in the plant tissue.

As used herein, the term “polypeptide” means a chain of at least two amino acids joined by peptide bonds. The chain may be linear, branched, circular or combinations thereof. Preferably, polypeptides are from about 10 to about 1000 amino acids in length, more preferably 10-50 amino acids in length. The polypeptides may contain amino acid analogs and other modifications, including, but not limited to glycosylated or phosphorylated residues.

As used herein, the term “recombinant polynucleotide” refers to a polynucleotide that has been altered, rearranged or modified by genetic engineering. Examples include any cloned polynucleotide, and polynucleotides that are linked or joined to heterologous sequences. Two polynucleotide sequences are heterologous if they are not naturally found joined together. The term recombinant does not refer to alterations to polynucleotides that result from naturally occurring events, such as spontaneous mutations.

As used herein, the terms “promoter” or “regulatory DNA sequence” means an untranslated DNA sequence which assists in, enhances, or otherwise affects the transcription, translation or expression of an associated structural DNA sequence which codes for a protein or other DNA product. The promoter DNA sequence is usually located at the 5′ end of a translated DNA sequence, typically between 20 and 100 nucleotides from the 5′ end of the translation start site.

As used herein, the term “RNA” means ribonucleic acid and the term “mRNA” means messenger RNA.

As used herein, the term “transgenic” refers to any cell, tissue, organ or organism that contains all or part of at least one recombinant polynucleotide. In many cases, all or part of the recombinant polynucleotide is stably integrated into a chromosome or stable extra-chromosomal element, so that it is passed on to successive generations.

Use of the term “transgenic” also refers to foreign polynucleotides. Use of the term “foreign” in this context refers to polynucleotides that may or may not have been altered, rearranged or modified or even that they have also preexisted within the cell, tissue, organ or organism. As illustration, a foreign polynucleotide is any polynucleotide that is introduced or re-introduced into a cell, tissue, organ or organism regardless of whether the same or a similar polynucleotide has already preexisted there. Any addition of any polynucleotide to a cell, tissue, organ or organism is considered by the present invention to be encompassed by the term “transgenic.”

As used herein, the term “transgenic plant” refers to any plant, plant cell, callus, plant tissue or plant part, that contains all or part of at least one recombinant polynucleotide. In many cases, all or part of the recombinant polynucleotide is stably integrated into a chromosome or stable extra-chromosomal element, so that it is passed on to successive generations.

As used herein, the term “transgenic plant material” is any plant matter, including, but not limited to cells, protoplasts, tissues, leaves, seeds, stems, fruits and tubers both natural and processed, containing a recombinant polynucleotide. Further, plant material includes processed derivatives thereof including, but not limited to food products, food stuffs, food supplements, extracts, concentrates, pills, lozenges, chewable compositions, powders, formulas, syrups, candies, wafers, capsules and tablets.

Hexosamine Biosynthetic Pathway Genes

Plants are not known to synthesize hexosamines, such as glucosamine or galactosamine, in nutritionally or commercially useful amounts. It is believed, however, that one or more foreign genes can be provided to plant cells, and to the cells of other organisms that do not normally synthesize hexosamines in appreciable amounts, in order to provide a complete and functioning pathway for the biosynthesis of a hexosamine. The present invention is considered to include the provision of a hexosamine biosynthetic pathway for organisms that are genotypically negative for hexosamine biosynthesis, and also is considered to cover the addition of recombinant polynucleotides encoding certain enzymes of the hexosamine synthesis pathway to organisms that already have an endogenous hexosamine biosynthetic pathway. Increased and localized expression of hexosamines are advantages that the present invention could add to organisms that already have such an endogenous pathway.

In one aspect, the present invention involves engineering one or more metabolic pathways in a plant cell so that transgenic plants are capable of synthesizing hexosamine sugars from endogenous amino acid and carbohydrate precursors. The source of the appropriate genes for glucosamine synthesis, for example, are those species that synthesize glucosamine-6-phosphate or glucosamine. In preferred embodiments, the transgenic plant is capable of synthesizing a hexosamine in an edible part of the plant.

The practice of the present invention will employ, unless otherwise indicated, conventional techniques of molecular biology, microbiology, cell biology, and biochemistry, which are within the skill of the art. Such techniques are explained fully in the literature, such as, Sambrook et al., “Molecular Cloning: A Laboratory Manual”, second edition, Cold Spring Harbor Press, Cold Spring Harbor, N.Y. (1989).

Glucosamine (2-amino-2-deoxyglucose) is an amino sugar found generally in chitin (insects/crustaceans), cell membranes, and mucopolysaccharides (e.g., as a component of cartilage). It occurs naturally in mammals and is a precursor of the glycosaminoglycan disaccharide unit, a building block of articular cartilage. In natural biosynthetic pathways, these disaccharide units polymerize, via β-linkages, to form glycosaminoglycans (GAG), which act as building blocks of articular cartilage. GAGs are long, negatively charged carbohydrate chains composed of repeating disaccharide units.

The two main GAGs are chondroitin sulfate and keratin sulfate. Chondroitin sulfate is composed of repeating disaccharide units that include glucuronic acid and galactosamine. Keratin sulfate (or keratosulfate) is composed of repeating disaccharide units composed of galactose and N-acetyl-D-glucosamine-6-sulfate. Because of their negative charge, GAGs are bound to other matrix components and cell adhesion molecules and are covalently linked to a core protein to form proteoglycans (PGs). See Ruoslahti, E., Annu. Rev. Cell Biol. 4:229-255 (1988). Proteoglycans are then assembled along with collagen fibers into a matrix to form resilient connective tissues such as cartilage.

One known hexosamine biosynthetic pathway begins at the glycolysis pathway intermediate, fructose-6-phosphate. From there, the enzyme glutamine:fructose-6-phosphate amidotransferase (EC 2.6.1.16)(“GFAT”), which is encoded by the polynucleotide sequence shown as SEQ ID NO: 1, and which includes the polypeptide sequence shown as SEQ ID NO: 2, transfers an amino group from glutamine or exogenous glucosamine and converts fructose-6-phosphate into glucosamine-6-phosphate. GFAT is the rate-limiting step in the hexosamine biosynthetic pathway. It is down-regulated by accumulation of the final chemical intermediary for glucosamine biosynthesis, n-acetyl-glucosamine. See, e.g., Sayeski et al., Nucleic Acids Research, 25 (7):1458-1466 (1997). The present invention contemplates the provision of glucosamine without a phosphate group, but it is still within the scope of the invention to simply introduce GFAT into a cell, tissue or organism and produce glucosamine-6-phosphate as a hexosamine of the present invention. In the same way, each step of the hexosamine biosynthetic pathway, beginning with GFAT, provides a potential stopping point for the production of hexosamines in a cell, tissue, organ, or organism.

In a preferred embodiment, an enzyme capable of cleaving the phosphate group from glucosamine-6-phosphate molecule would be employed as a second step. This would be desirable due to the additional complexity of introducing increasing numbers of genes. Currently, however, no glucosamine-6-phosphatase is known to the inventor. It is still within the scope of the present invention, however, that if such an enzyme is developed or discovered, it could be used along with the GFAT enzyme to produce glucosamine in a transgenic cell, tissue or organism. Until such a time as a phosphatase is discovered to remove the phosphate group from glucosamine-6-phosphate, the enzymes of the recognized biosynthetic pathway may be utilized instead.

As an alternative, other schemes may be used to provide a multi-step system for glucosamine production. As a general category, phosphatase enzymes are capable of cleaving phosphate groups. Many organisms are known to have enzymes such as glucose-6-phosphatase (EC 3.1.3.9), which is encoded by the polynucleotide sequence shown in SEQ ID NO: 13, and which includes the polypeptide sequence shown in SEQ ID NO: 14, that can remove phosphate groups from glucose-6-phosphate. It is foreseeable by the present invention that such an enzyme (or a variant or mutant created by mutagenesis of an existing sugar phosphatase) may also work to cleave the phosphate group from glucosamine-6-phosphate, thus yielding glucosamine.

A second step in hexosamine biosynthesis uses the enzyme glucosamine phosphate n-acetyltransferase (EC 2.3.1.4), which is encoded by the polynucleotide sequence shown in SEQ ID NO: 3, and which includes the polypeptide sequence shown in SEQ ID NO: 4, and acetyl-CoA group to add an acetyl group to glucosamine-6-phosphate thereby converting it to n-acetyl-glucosamine-6-phosphate. In a third step, the enzyme phosphoacetylglucosamine mutase (EC 5.4.2.3), which is encoded by the polynucleotide sequence shown in SEQ ID NO: 5, and which includes the polypeptide sequence shown in SEQ ID NO: 6, rearranges the phosphate group around to a different carbon on the glucosamine backbone and forms n-acetyl-glucosamine-1-phosphate. In a fourth step, the enzyme UDP-n-acetyl-glucosamine pyrophosphorylase (EC 2.7.7.23), which is encoded by the polynucleotide sequence shown in SEQ ID NO: 7, and which includes the polypeptide sequence shown in SEQ ID NO: 8, removes the phosphate group from n-acetyl-glucosamine-1-phosphate and adds a uridine diphosphate group from UTP forming UDP-n-acetyl-glucosamine.

After the formation of UDP-n-acetyl-glucosamine, the pathway branches into the formation of two more aminosugars: galactosamine and mannosamine. In a fifth step, the enzyme UDP-n-acetyl-glucosamine-4-epimerase (EC 5.1.3.7), which is encoded by the polynucleotide sequence shown in SEQ ID NO: 9, and which includes the polypeptide sequence shown in SEQ ID NO: 10, converts UDP-n-acetyl-glucosamine into UDP-n-acetyl-galactosamine. In a sixth step, the enzyme UDP-n-acetyl-glucosamine-2-epimerase (EC 5.1.3.14), which is encoded by the polynucleotide sequence shown in SEQ ID NO: 11, and which includes the polypeptide sequence shown in SEQ ID NO: 12, converts UDP-n-acetyl-glucosamine into UDP-n-acetyl-mannosamine.

In the biosynthetic pathway of the present invention, the hexosamine biosynthetic pathway may be stopped at any point or step before the final end-product. Thus, for example, for purposes of the present invention, any of: UDP-n-acetyl-glucosamine, n-acetyl-glucosamine, glucosamine-6-phosphate, n-acetyl-glucosamine-6-phosphate, n-acetyl-glucosamine-1-phosphate, glucosamine-1-phosphate, glucosamine, glucosamine sulfate and glucosamine hydrochloride are considered to provide glucosamine, which is useful for the purposes described herein. It should also be understood to be within the scope of the present invention that, once a hexosamine biosynthetic product is produced in a transgenic organism, further chemical treatments or modifications to any product of the pathway may be performed and yet still fall within the scope of the present invention.

Methods for Obtaining Hexosamine Pathway Genes

Several different ways are known in the art to obtain (or “clone”) cDNA's or genomic clones of the aforementioned hexosamine pathway genes of interest. Any of the known cloning methods can be used in the present invention. For general information, see, Sambrook et al., Ibid. With the use of methods such as those described in that reference, a glucosamine-6-phosphatase may be found, or created by mutating a known phosphatase, as discussed above, for use with the present invention, as may be other known or unknown hexosamine pathway genes. For example, short fragments of the full length genes of the present invention may be used as a hybridization probe for a cDNA library. The short fragment sequence information is obtainable from the art. Short sequences called oligonucleotides or probes can be synthesized to have sequence complementarity to the hexosamine pathway gene desired. Radiolabeled oligonucleotides having a sequence complementary to that of the gene of the present invention are used to screen a library of, for example, crustacean cDNA, genomic DNA or mRNA to determine to which members of the library the probes hybridize. The probes are then used to isolate the full length cDNA from a cDNA library prepared from total RNA of an organism such as a crustacean. Probes of this type preferably have at least 30 bases and may contain, for example, 50 or more bases. The probe may also be used to identify a cDNA clone corresponding to a full length transcript and a genomic clone or clones that contain the complete gene including regulatory and promotor regions, exons, and introns. After successful hybridization of a cDNA library clone, restriction enzyme mapping or direct sequencing can confirm the identity of the isolated clone.

Alternatively, short oligonucleotide DNA sequences may be synthesized to have sequence homology to 5′ and 3′ ends of a published hexosamine biosynthetic pathway gene. Polymerase Chain Reaction (PCR) may then be employed to isolate the genetic sequence between the two oligonucleotides from total genomic DNA of an organism.

For the present invention, sources of polynucleotides for cloning the hexosamine biosynthetic pathway include bacteria, fungi, insects, mollusks, sharks, crustaceans, and mammals. Preferably, the hexosamine biosynthetic pathway is cloned from crustaceans such as lobster, crab, or shrimp.

Vectors and Expression Cassettes

One embodiment of the present invention includes a recombinant vector, which includes at least one isolated nucleic acid molecule of the present invention inserted into any vector capable of delivering the nucleic acid molecule into a cell. Such a vector contains heterologous nucleic acid sequences, that is, nucleic acid sequences that are not naturally found adjacent to nucleic acid molecules of the present invention and that preferably are derived from a species other than the species from which the nucleic acid molecule(s) are derived. The vector can be either RNA or DNA, and can be either prokaryotic or eukaryotic. Recombinant vectors can be used in the cloning, sequencing, and/or otherwise manipulation of hexosamine biosynthetic pathway nucleic acid molecules of the present invention.

One type of recombinant vector, referred to herein as a recombinant molecule, comprises a nucleic acid molecule of the present invention operatively linked to control sequences within an expression vector. The phrase “operatively linked” refers to insertion of a nucleic acid molecule into an expression vector in a manner such that the molecule is able to be expressed when transformed into a cell. As used herein, an expression vector is a DNA or RNA vector that is capable of transforming a cell and of effecting expression of a specified nucleic acid molecule. Preferably, the expression vector is also capable of replicating within the cell. Expression vectors of the present invention include any vectors that function (i.e., direct gene expression) in recombinant cells of the present invention, including in bacterial, fungal, yeast, crustacean, mammalian, insect, other animal, and plant cells.

Preferred expression vectors of the present invention can direct gene expression in plant cells.

In preferred embodiments, expression vectors of the present invention contain regulatory sequences such as transcription control sequences, translation control sequences, origins of replication, and other regulatory sequences that are compatible with the recombinant cell and that control the expression of nucleic acid molecules of the present invention. In particular, recombinant molecules of the present invention include transcription control sequences. Transcription control sequences are sequences that control the initiation, elongation, and termination of transcription. Particularly important transcription control sequences are those that control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art.

The present invention also provides vectors containing the expression cassettes of the invention. By “vector”, what is meant is a polynucleotide sequence that is able to replicate in a host cell, but such definition also includes transient vectors that could be used to introduce DNA by, for example, homologous recombination in the genome, which do not replicate in a host cell. The vector can comprise DNA or RNA and can be single or double stranded, and linear or circular. Various plant expression vectors and reporter genes are described in Gruber et al. in Methods in Plant Molecular Biology and Biotechnology, Glick et al., eds, CRC Press, pp.89-119 (1993); and Rogers et al., Meth Enzymol 153:253-277 (1987). Standard techniques for the construction of the vectors of the present invention are well-known to those of ordinary skill in the art and can be found in such references as Sambrook et al., Ibid.

The following vectors, which are commercially available, are provided by way of example. Among vectors preferred for use in bacteria are pQE70, pQE60 and pQE-9, available from Qiagen; pBS, pD10, phagescript, psiX174, pbluescript SK, pbsks, pNH8A, pNH16a, pNH18A, pNH46A, available from Stratagene; and ptrc99a, pKK223-3, pKK233-3, pDR540, pRIT5 available from Pharmacia. These vectors are listed solely by way of illustration of the many commercially available and well known vectors that are available to those of skill in the art for use in accordance with this aspect of the present invention. It will be appreciated that any other plasmid or vector suitable for, for example, introduction, maintenance, propagation or expression of a polynucleotide or polypeptide of the invention in any host cell or tissue may be used in this aspect of the invention.

The vectors of the invention can contain 5′ and 3′ regulatory sequences necessary for transcription and termination of the polynucleotide of interest. Thus, the vectors can include a promoter and a transcriptional terminator. Other functional sequences may be included in the vectors of the inventions. Such functional sequences include, but are not limited to, introns, enhancers and translational initiation and termination sites and polyadenylation sites. Preferably, the expression cassettes of the present invention are engineered to contain a constitutive promoter 5′ to its translation initiation codon (ATG) and a poly(A) addition signal (AATAAA) 3′ to its translation termination codon. The control sequences can be those that can function in at least one plant, plant cell, plant tissue, or plant organ. These sequences may be derived from one or more genes, or can be created using recombinant technology.

Other elements such as introns, enhancers, polyadenylation sequences and the like, may also be a part of the recombinant DNA. Such elements may or may not be necessary for the function of the DNA, but may provide improved expression of the mRNA by affecting transcription, stability, or the like. For example, the maize AdhlS first intron may be placed between the promoter and the coding sequence in a particular recombinant DNA construction. This intron, when included in a DNA construction, is known to increase production of a protein in maize cells. (J. Callis et al., Genes and Develop., 1:1183 (1987)). However, sufficient expression for a selectable marker to perform satisfactorily can often be obtained without an intron. See T. Klein et al., Plant Physiol., 91:440 (1989). An example of an alternative suitable intron is the shrunken-l first intron of Zea mays.

Promoter Selection

The 5′ regulatory sequences (promoters) which are often used in creation of chimeric genes for plant transformation may cause either nominally constitutive expression in all cells of the transgenic plant, or regulated gene expression where only specific cells, tissues, or organs show expression of the introduced genes. For the present invention, it is a preferred embodiment to select a tissue-specific promoter for a host plant so that the hexosamines are expressed in the desired edible part(s) of the plant.

Promoters useful in the expression cassettes of the invention include any promoter that is capable of initiating transcription in a cell. Promoters which are known or found to cause transcription of a foreign gene in plant cells can be used in the present invention. Such promoters may be obtained from plants or viruses and include, but are not necessarily limited to the constitutive 35S promoter of cauliflower mosaic virus (CaMV) (as used herein, the phrase “CaMV 35S” promoter, includes variations of CaMV 35S promoter such as the 2× CaMV 35S promoter). See Odell et al., Nature 313:810-812 (1985). Constitutive promoters such as the 35S promoter are active under most conditions.

Examples of constitutive promoters that are useful in the present invention include the Sep1 promoter, the rice actin promoter (McElroy et al., Plant Cell 2:163-171 (1990)), the Arabidopsis actin promoter, the ubiquitan promoter (Christensen et al., Plant Molec Biol 18:675-689 (1989)); the pEmu promoter (Last et al., Theor Appl Genet 81:581-588 (1991)), the figwort mosaic virus 35S promoter, the Smas promoter (Velten et al., EMBO J 3:2723-2730 (1984)), the GRP1-8 promoter, the cinnamyl alcohol dehydrogenase promoter (U.S. Pat No. 5,683,439), promoters from the T-DNA of Agrobacterium, such as those from the mannopine synthase, nopaline synthase, and octopine synthase genes, the small subunit of ribulose bisphosphate carboxylase (ssuRUBISCO) promoter, and the like.

Developmental stage-preferred promoters are preferentially expressed at certain stages of development. Tissue and organ preferred promoters include those that are preferentially expressed in certain tissues or organs, such as fruits, leaves, roots, stems, seeds, or xylem. Examples of tissue preferred and organ preferred promoters include, but are not limited to Prha (expressed in root, seedling, lateral root, shoot apex, cotyledon, petiole, inflorescence stem, flower, stigma, anthers, and silique, and auxin-inducible in roots); VSP2 (expressed in flower buds, flowers, and leaves, and wound inducible); SUC2 (expressed in vascular tissue of cotyledons, leaves and hypocotyl phloem, flower buds, sepals and ovaries); AAP2 (silique-preferred); SUC1 (anther and pistil preferred); AAP1 (seed preferred); Saur-AC1 (auxin inducible in cotyledons, hypocotyl and flower); Enod 40 (expressed in root, stipule, cotyledon, hypocotyl and flower); and VSP1 (expressed in young siliques, flowers and leaves).

Preferably, the present invention may utilize promoters for genes which are known to give high-level expression in edible plant parts, such as the tuber-specific patatin gene promoter from potato. For further information, see, e.g., H. C. Wenzler, et al, Plant Mol. Biol., 12:41-50 (1989). It is also known that a tissue-specific promoter for one species may be used successfully in directing transgenic DNA expression in specific tissues of other species. For example, the potato tuber promoters will also function in tomato plants to cause fruit specific expression of an introduced gene. See U.S. Pat. No. 08/344,639, to Barry et al.

A further example of a tissue-specific promoter is the seed specific bean phaseolin and soybean beta-conglycinin promoters. See, e.g., Keeler et al., Plant. Mol. Biol. 34:15-29 (1997).

Examples of other promoters for use with a preferred embodiment of the present invention include fruit-specific promoters such as the E8 promoter, described in Deikman et al., EMBO J. 2:3315-3320 (1998). The activity of the E8 promoter is not limited to tomato fruit, but is thought to be compatible with any system wherein ethylene activates biological process, including fruit ripening. See U.S. Pat. No. 5,234,834 to Fischer et al. Other promoters suitable for use with the present invention are the MADS-box promoters, endo-β-1,4-glucanase promoter, expansin promoters, egase promoters, pectate lyase promoter, polygalacturonase promoters, and ethylene biosynthesis promoters.

Also useful in the present invention are: (a) another tomato fruit-specific promoter, LeExp-1 (See U.S. Pat. No. 6,340,748); (b) the banana fruit-specific promoters known as the MT clones (See U.S. Pat. No. 6,284,946); and (c) several strawberry specific promoters termed GSRE2, GSRE49, SEL1, and SEL2 (See U.S. Pat. No. 6,080,914). For purposes of the present invention any tissue-specific or fruit-specific promoter can be used to direct the expression and, therefore, products of the hexosamine biosynthetic pathway genes into the edible part(s) of a plant.

Further examples of suitable plants and, therefore promoters, include: raspberry, pear, apple, kiwifruit, orange, watermelon, and carrots, among others.

Terminators and Selectable Markers

The recombinant polynucleotides that are to be introduced into the plant cells will preferably contain either a selectable marker or a reporter gene, or both, in order to facilitate identification and selection of transformed cells. Alternatively, the selectable marker can be carried on a separate piece of DNA and used in a co-transformation procedure. Both selectable markers and reporter genes can be flanked with appropriate regulatory sequences to enable expression in plants. Useful selectable markers are well known in the art and include, for example, antibiotic and herbicide resistance genes. Examples of such markers include genes encoding drug or herbicide resistance, such as hygromycin resistance (hygromycin phosphotransferase (HPT)), spectinomycin resistance (encoded by the aada gene), kanamycin and gentamycin resistance (neomycin phosphotransferase (nptll)), streptomycin resistance (streptomycin phosphotransferase gene (SPT)), phosphinothricin or basta resistance (barnase (bar)), chlorsulfuron reistance (acetolactase synthase (ALS)), chloramphenicol resistance (chloramphenicol acetyl transferase (CAT)), G418 resistance, lincomycin resistance, methotrexate resistance, glyphosate resistance, and the like.

The expression cassettes of the invention may be covalently linked to genes encoding enzymes that are easily assayed, for example, luciferase, alkaline phosphatase, B-galactosidase (B-gal), B-glucuronidase (GUS), and the like.

Transcriptional termination regions include, but are not limited to, the terminators of the octopine synthase and nopaline synthase genes in the A. tumefaciens Ti plasmid. For further information, see Ballas et al., Nuc Acid Res 17:7891-7903 (1989). If translation of the transcript is desired, translational start and stop codons can also be provided.

Organism Choice

Since many edible plants that are used by mammals for food are dicotyledenous and monocotyledenous plants, both dicotyledon cells and monocotyledon cells can be used as a host organism in the present invention. Monocotyledon transformation is applicable, especially in the production of certain grains, e.g. corn, wheat and rice, as may be useful when it is desired that the product hexosamine be particularly palatable for humans and companion animals. However, it is foreseeable by one of ordinary skill in the art that a variety of organisms other than plants could also be transformed with the hexosamine pathway genes in order to produce hexosamines. Therefore, cells of the present invention can be any cell capable of producing at least one hexosamine of the present invention, and include bacterial, fungal (yeasts, in particular), insect, crustacean, mammalian and plant cells. Preferred cells for transformation by hexosamine pathway nucleic acid molecules of the present invention include plant, bacterial, fungal, crustacean, insect and mammalian cells, and plant cells are more preferred.

For plant transformation systems, the host plant selected for genetic transformation preferably has edible tissue in which the amino sugar hexosamine can be synthesized. Thus, the hexosamine is expressed in a part of the plant, such as the fruit, leaves, stems, seeds, or roots, which may be consumed by a mammal for which the hexosamine is intended. In an alternate embodiment, the hexosamine may be produced in any part of the plant, including in a nonedible plant part, and then isolated and/or purified. The isolated and/or purified hexosamine can then be administered to a mammal by one of various known methods of administering medications or nutraceuticals.

Various other considerations are made in selecting the host plant. For example, it may sometimes be preferred that the edible tissue of the host plant not require heating prior to consumption. Also, since certain medications may be more easily tolerated when in liquid form, it may sometimes be preferred that the host plant express hexosamine which will function as a nutraceutical in the form of a drinkable liquid.

Plants which are suitable for the practice of the present invention include any gymnosperm, dicotyledon and monocotyledon. Preferred plants are those which are edible in part or in whole by a human or an animal. Edible plants that may be useful in the present invention are not particularly limited and may be gymnosperms, monocots and dicots. Such plants include cereals (wheat, barley, rye, oats, rice, sorghum, related crops, etc.), beet, pear-like fruits, stone fruits, and soft fruits (apple, pear, plum, peach, Japanese apricot, prune, almond, cherry, strawberry, raspberry, black berry, tomato, pepper, etc.), legumes (kidney bean, lentil, pea, soybean), oil plants (rape, canola, mustard, poppy, olive, sunflower, coconut, castor, cocoa bean, peanut, soybean, corn, etc.), Cucurbitaceae (pumpkin, cucumber, melon, etc.), citrus (orange, lemon, grape fruit, mandarin, Watson pomelo (citrus natsudaidai), etc.), vegetables (lettuce, cabbage, celery cabbage, Chinese radish, carrot, onion, potato, etc.), camphor trees (avocado, cinnamon, camphor, etc.), corn, tobacco, nuts, coffee, sugar cane, tea, grapevine, hop and banana.

Edible plants that are particularly useful include rice, wheat, barley, rye, corn, potato, carrot, sweet potato, sugar beet, bean, pea, chicory, lettuce, cabbage, cauliflower, broccoli, turnip, radish, spinach, asparagus, onion, garlic, eggplant, pepper, celery, canot, squash, pumpkin, zucchini, cucumber, apple, pear, quince, melon, plum, cherry, peach, nectarine, apricot, strawberry, grape, raspberry, blackberry, pineapple, avocado, papaya, mango, banana, soybean, tomato, sorghum and raspberries, banana and other such edible varieties.

It is particularly advantageous in certain disease treatment protocols for mammals to produce the hexosamines in a juice for ease of administration to humans. Examples of such consumable juices include, without limitation, tomato juice, soybean milk carrot juice, or a juice made from one or a variety of fruit or berry types. Other foodstuffs for easy consumption might include dried fruit.

Methods for Transforming the Hexosamine Pathway Genes into an Organism

Methods and compositions for transforming a bacterium, a fungal cell, a plant cell, or an entire plant with one or more expression vectors comprising a hexosamine biosynthesis protein-encoding gene segment are further aspects of this disclosure. A transgenic bacterium, fungal cell (such as, for example, a yeast cell), plant cell or plant derived from such a transformation process or the progeny and seeds from such a transgenic plant are also further embodiments of the invention. A variety of techniques are available for the introduction of the genetic material into or transformation of the plant cell host. However, the particular manner of introduction of the plant vector into the host is not critical to the practice of the present invention, and any method which provides for efficient transformation can be employed.

Methods for the introduction of polynucleotides into plants and for generating transgenic plants are known to those skilled in the art. See e.g. Weissbach & Weissbach (1988) Methods for Plant Molecular Biology, Academic Press, N.Y.; Grierson & Corey (1988).

The choice of plant tissue source or cultured plant cells for transformation will depend on the nature of the host plant and the transformation protocol. Useful tissue sources include callus, suspension culture cells, protoplasts, leaf segments, root segments, stem segments, tassels, pollen, embryos, hypocotyls, tuber segments, meristematic regions, and the like. In preferred embodiments, the tissue source is regenerable, in that it will retain the ability to regenerate whole, fertile plants following transformation.

In a preferred embodiment of the present invention, the Agrobacterium-Ti plasmid system is utilized. Agrobacterium tumefaciens-meditated transformation techniques are well described in the scientific literature. See, e.g., Horsch et al., Science 233: 496-498 (1984). Descriptions of the Agrobacterium vector systems and methods for Agrobacterium-mediated gene transfer are provided in Gruber et al., supra. Although Agrobacterium is useful primarily in dicots, certain monocots can also be transformed by Agrobacterium. For instance, Agrobacterium transformation of maize is described in U.S. Pat. No. 5,550,318.

Agrobacterium-mediated transformation utilizes A. tumefaciens, the etiologic agent of crown gall, a disease of a wide range of dicotyledons and gymnosperms that results in the formation of tumors or galls in plant tissue at the site of infection. Agrobacterium, which normally infects the plant at wound sites, carries a large extrachromosomal element called Ti (tumor-inducing) plasmid.

Ti plasmids contain two regions required for tumor induction. One region is the T-DNA (transfer-DNA) which is the DNA sequence that is ultimately found stably transferred to plant genomic DNA. The other region is the vir (virulence) region which has been implicated in the transfer mechanism. Although the vir region is absolutely required for stable transformation, the vir DNA is not actually transferred to the infected plant. Transformation of plant cells mediated by infection with A. tumefaciens and subsequent transfer of the T-DNA alone have been well documented. See, e.g., Bevan, M. W. et al., Int. Rev. Genet, 16: 357 (1982).

The construction of an Agrobacterium transformation vector system has two elements. First, a plasmid vector is constructed which replicates in Escherichia coil (E. coli). This plasmid contains the DNA encoding the protein of interest (in this invention a member of the hexosamine biosynthetic pathway). This DNA is flanked by T-DNA border sequences that define the points at which the DNA integrates into the plant genome. Border sequences include both a left border (LB) and a right border (RB).

Usually a gene encoding a selectable marker (such as a gene encoding resistance to an antibiotic or herbicide such as hygromycin or Basta) is also inserted between the left border and right border sequences. The expression of this gene in transformed plant cells gives a positive selection method to identify those plants or plant cells which have an integrated T-DNA region. The second element of the process is to transfer the plasmid from E. coli to Agrobacterium. This can be accomplished via a conjugation mating system, or by direct uptake of plasmid DNA by Agrobacterium using such methods as electroporation.

Those skilled in the art would recognize that there are multiple choices of Agrobacterium strains and plasmid construction strategies that can be used to optimize genetic transformation of plants. They will also recognize that A. tumefaciens may not be the only Agrobacterium strain used. Other Agrobacterium strains such as A. rhizogenes might be more suitable in some applications. See Lichtenstein and Fuller in: Genetic Engineering, Volume 6, Ribgy (ed) Academic Press, London (1987).

The present invention is not limited to the Agrobacterium-Ti plasmid system, and includes any direct physical method of introducing foreign DNA into an organism such as plant cells. Direct transformation involves the uptake of exogenous genetic material into cells or protoplasts. Such uptake may be enhanced by use of chemical agents or electric fields. See Dewulf J. and Negrutiu I., Direct gene transfer into protoplasts: The chemical approach, in: A Laboratory Guide for Cellular and Molecular Plant Biology. Eds. I. Negrutiu and G. Gharti-Chhetri. Birkhauser Verlag. Basel (1991). The exogenous genetic material may then be integrated into the nuclear genome. For mammalian cells, transfection procedures can be used. For bacterial cells, electroporation or heat shock methods can be employed.

For plant systems, direct gene transfer can also be accomplished by polyethylene glycol (PEG) mediated transformation. This method relies on chemicals to mediate the DNA uptake by protoplasts and is based on synergistic interactions between Mg⁺², PEG, and possibly Ca⁺². See, e.g., Negrutiu, R. et al., Plant Mol. Biol., 8: 363 (1987). Alternatively, exogenous DNA can be introduced into cells or protoplasts by microinjection. In this technique, a solution of the plasmid DNA or DNA fragment is injected directly into the cell with a finely pulled glass needle.

Another procedure for direct gene transfer involves bombardment of cells by micro-projectiles carrying DNA. In this procedure, commonly called particle bombardment or biolistics, tungsten or gold particles coated with the exogenous DNA are accelerated toward the target cells. The particles penetrate the cells carrying with them the coated DNA. Microparticle acceleration has been successfully demonstrated to give both transient expression and stable expression in cells suspended in cultures, protoplasts, immature embryos of plants, including, but not limited to, onion, maize, soybean, and tobacco. Microprojectile transformation techniques are described in Klein T. M., et al, Nature, 327: 70-73 (1987).

Electric fields may also be used to introduce genetic material into the cells of an organism. The application of brief, high-voltage electric pulses to a variety of bacterial, animal and plant cells leads to the formation of nanometer-sized pores in the plasma membrane. DNA is taken directly into the cell cytoplasm either through these pores or as a consequence of the redistribution of membrane components that accompanies closure of the pores. Electroporation techniques are described in Fromm et al., Proc. Natl. Acad. Sci. 82: 5824 (1985).

Viral means of introducing DNA into cells are known in the art. In particular, a number of viral vector systems are known for the introduction of foreign or native genes into mammalian cells. These include the SV40 virus (See, e.g., Okayama et al., Molec. Cell Biol. 5:1136-1142 (1985)); bovine papilloma virus (See, e.g., DiMaio et al., Proc. Natl. Acad. Sci. USA 79:4030-4034 (1982)); adenovirus (See, e.g., Morin et al., Proc. Natl. Acad. Sci. USA 84:4626 (1987)). For further information regarding viral vector systems, see, e.g., Yifan et al., Proc. Natl. Acad. Sci. USA 92:1401-1405 (1995).

A number of viral vector systems are also known for the introduction of foreign or native genes into plant cells. A variety of plant viruses that can be employed as vectors are known in the art and include cauliflower mosaic virus (CaMV), geminivirus, brome mosaic virus, and tobacco mosaic virus.

Once plant cells have been transformed, there are a variety of methods for regenerating plants, as is well known in the art. See, e.g., Weissbach, et al., supra. The particular method of regeneration will depend on the starting plant tissue and the particular plant species to be regenerated. Plant regeneration from cultured protoplasts is described in Evans et al., Protoplasts Isolation and Culture, Handbook of Plant Cell Culture, Macmillilan Publishing Company, New York, pp.124-176 (1983). In recent years, it has become possible to regenerate many species of plants from callus tissue derived from plant explants. Regeneration of plants from tissue transformed with A. tumefaciens has been demonstrated for several species of plants. These include, but are not limited to, sunflower, tomato, white clover, rapeseed, cotton, tobacco, potato, maize, rice, and numerous vegetable crops.

After a transgenic plant has been obtained, it may be used as a parent to produce progeny plants and plant lines. Conventional plant breeding methods can be used, including, but not limited to crossing and backcrossing, self-pollination and vegetative propagation. Techniques for breeding plants are known to those skilled in the art. The progeny of a transgenic plant are included within the scope of the invention, provided that the progeny contain all or part of the transgenic construct.

Isolation or use of Hexosamines from Transgenic Organisms

The present invention provides for the production of hexosamines by transformed plant cells and by transformed organisms other than plant cells. Any organism in which the polynucleotides may express their corresponding polypeptides for the hexosamine biosynthetic pathway, is also considered to be within the scope of the invention. In one embodiment, a bacterial, crustacean, insect, mollusk, shark, fungal, or mammalian cell line is used as a bioreactor for the expression of the 1) hexosamine pathway polypeptides, 2) synthesis of hexosamines, and 3) subsequent purification of said hexosamines, such as, for example, for glucosamine and/or galactosamine production. Plant cell lines would also be considered as capable of being bioreactors within the present invention. In the case of cell bioreactors, the subject hexosamine could be produced either intracellularly secreted into the periplasmic space or Gram-negative bacteria, or excreted into the culture medium.

Once the hexosamines are purified from a bioreactor production system, they may be encapsulated or prepared in tablet form for mammalian consumption. Alternatively, they may be introduced into edible foods or beverages as nutraceutical treatments. In addition, these purified hexosamines may be combined into edible foods, beverages, capsules or tablets with other materials. The other materials can be either therapeutically useful compounds, or they may be colorants, fillers, anti-oxidants, viscosity-enhancers, absorbents, surfactants, and the like.

An embodiment of the present invention includes the use of a complete living organism, such as a plant. Harvesting these plants and purifying the expressed hexosamines from any of the plant parts would be considered to correspond within the teachings of the present invention.

There may be plant, mammalian, crustacean, bacterial, or fungal cell cultures in vitro, for example, in liquid medium. For the cell types of the present invention, which can be used in the method of hexosamine production, at least three main types of bioreactors may be used for the bioproduction reactions: the batch tank, the packed bed and the continuous-flow stirred tank. Applications and characteristics of these systems have been reviewed by M. D. Lilly in “Recent Advances in Biotechnology”, eds. F. Vardar-Sukan.

The “batch” cultures are comparable to those carried out in an Erlenmeyer flask. Since the medium is not changed, these cells have only a limited quantity of nutrient materials. The “fed batch” culture corresponds, for its part, to a “batch” culture with programmed addition of substrate, or other nutrients. In a continuous culture, the cells are supplied continuously with nutrient medium and an equal volume of the biomass-medium mixture is removed in order to maintain the volume of the reactor(s) constant.

Isolated hexosamines of the present invention can be produced in a variety of ways, including production and recovery of naturally-occurring hexosamines, production and recovery of recombinantly made hexosamines, and chemical synthesis of the hexosamines. In one embodiment, a hexosamine of the present invention is produced by culturing a cell capable of expressing the hexosamine biosynthetic pathway proteins under conditions effective to produce the proteins, and finally recovering and purifying hexosamines that are produced by the biosynthetic hexosamine pathway.

A preferred cell for this type of culture is a recombinant cell of the present invention. Effective culture conditions include, but are not limited to, effective media, bioreactor, temperature, pH and oxygen conditions that permit hexosamine pathway protein synthesis and hexosamine production. An effective medium refers to any medium in which a cell is cultured to produce a hexosamine of the present invention. Such medium typically comprises an aqueous medium having assimilable carbon, nitrogen and phosphorous sources, and other appropriate salts, minerals, metals and nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

Depending on the vector and transformed cell system used for production, resultant hexosamines of the present invention may either remain within the recombinant cell; be secreted into the fermentation medium; be secreted into a space between two cellular membranes, such as the periplasmic space in E. coli, or be retained on the outer surface of a cell or viral membrane. Suitable purification techniques are known in the art.

It is also known that plants have the capacity to act as “natural” factories for the inexpensive production of biopharmaceutical proteins. See Giddings et al., Nat Biotechnol 18(11):1151-1155 (2000). Plants are well-suited for the production of biopharmaceuticals because they are typically relatively easy and inexpensive to grow, harvest, and process.

As described above, plants have been genetically transformed with foreign DNA to produce a biopharmaceutical product. Moreover, such a transformed plant can sometimes be induced to produce a biopharmaceutical product in its edible parts. In this case, administration of the biopharmaceutical to a subject can be as simple as allowing the subject to eat the edible plant parts, thereby avoiding a time-consuming and inefficient extraction or purification step.

Methods have been successfully demonstrated to transform plants in a stable manner. Such methods include, among others; Agrobacterium-mediated transformation, viral-mediated transformation, biolistics (microprojectile bombardment), protoplasts (PEG), electroporation, and microinjection. A typical procedure involves the mechanical, bacterial, or viral introduction of the foreign DNA, which encodes for the biosynthetic pathway or protein of interest, into immature plant parts. As the “transformed” immature plant develops, the foreign DNA becomes incorporated into the plant's own DNA. After incorporation, the foreign DNA is translated into the protein of interest and expressed in either the entire plant or solely in the edible parts.

In order for the protein of interest to be produced solely in the edible portions of the plant, a “tissue-specific” promoter is ordinarily incorporated along with the foreign DNA. The “tissue-specific” promoter is placed upstream to the foreign DNA and allows the plant to express the foreign DNA only in certain desired regions such as in the fruit or tuber. For stable expression of the protein of interest, the newly transformed plant is usually allowed to set seed so that it is the subsequent generations that are used for biopharmaceutical harvesting or for edible medications.

Many reported instances of genetically engineering plants for edible biopharmaceutical use involve transforming plants with foreign DNA encoding for bacterial or viral cell surface antigens. Once the “transformed” fruit or vegetable is ingested, the cell surface antigens in the fruit or vegetable initiate an immune response, effectively conveying an “edible” vaccine. For example, U.S. Pat. No. 6,136,320 to Arntzen et al., demonstrates transforming tomato plants with the Hepatitis B virus surface antigen for use as a vaccine in edible tomato juice. See also Kapusta et al., FASEB 13:1796-1799 (1999). Clearly, the production of biopharmaceuticals in edible plant parts would appear to simplify the process of obtaining, purifying, and ingesting the protein of interest in difficult to reach areas, especially, in third-world countries.

Genetically engineered plants have also been transformed with foreign DNA encoding for a fructosyltransferase enzyme that converts sucrose into oligosaccharides. See U.S. Pat. No. 6,147, 280 to Smeekens et al. The Smeekens et al. patent appears to describe the plant production of oligosaccharides when used for sugar-substituted food sweeteners. Other reported transformed edible plant uses have been for the production of insulin in potatoes and the manufacture of antibodies in soybeans against the genital herpes virus. See Mofat, Science 282 (5397):18 2176-2178 (1998).

In addition to plants, many other organisms have been reported as useful for genetic transformation and bioharvesting. For example, fungal, bacterial, and mammalian cell lines have been utilized to express bioproduction-related proteins and compounds.

In the present method, a subject in need of prevention, treatment, or inhibition of pain, inflammation or inflammation-associated disorder is treated with an amount of a hexosamine-containing material of the present invention that is sufficient to provide an amount of a hexosamine constituting a pain or inflammation suppressing treatment or prevention effective amount.

As used herein, an “effective amount” means the dose or effective amount to be administered to a patient and the frequency of administration to the subject which is readily determined by one having ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances.

As used herein, the term “therapeutically effective” is intended to qualify the amount of each agent for use in therapy which will achieve the goal of preventing, or improvement in the severity of, the disorder being treated, while avoiding adverse side effects typically associated with alternative therapies. An amount of hexosamine that causes a decrease in the frequency of incidence is “prophylactically effective”, where the term “prophylactic” refers to the prevention of disease, whereas the term “therapeutic” refers to the effective treatment of existing disease.

Those skilled in the art will appreciate that dosages may also be determined with guidance from Goodman & Goldman's The Pharmacological Basis of Therapeutics, Ninth Edition (1996), Appendix II, pp. 1707-1711.

In the present method, the amount of glucosamine that is used in the novel method of treatment preferably ranges from about 0.1 to about 500 milligrams per day per kilogram of body weight of the subject (mg/day.kg), more preferably from about 0.5 to about 100 mg/day kg, even more preferably from about 1 to about 50 mg/day.kg, yet more preferably from about 5 to about 35 mg/day.kg, and even more preferably from about 15 to about 25 mg/day.kg.

The subject hexosamine can be supplied in the form of a novel therapeutic composition that is believed to be within the scope of the present invention. The relative amounts of the hexosamine in the therapeutic composition may be varied and may be as described above. The hexosamines that are described above can be provided in the therapeutic composition so that the preferred amount of the compound is supplied by a single dosage, a single capsule for example, or, by up to four, or more, single dosage forms.

When the subject hexosamine is supplied along with a pharmaceutically acceptable carrier, a pharmaceutical composition is formed. A pharmaceutical composition of the present invention is directed to a composition suitable for the prevention or treatment of pain, inflammation and/or an inflammation-associated disorder. The pharmaceutical composition comprises a pharmaceutically acceptable carrier and a hexosamine. Pharmaceutically acceptable carriers include, but are not limited to, physiological saline, Ringer's solution, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically or prophylactically effective amount.

The term “pharmaceutically acceptable” is used herein to mean that the modified noun is appropriate for use in a pharmaceutical product. Pharmaceutically acceptable cations include met allic ions and organic ions. More preferred met allic ions include, but are not limited to, appropriate alkali met al salts, alkaline earth met al salts and other physiologically acceptable met al ions. Exemplary ions include aluminum, calcium, lithium, magnesium, potassium, sodium and zinc in their usual valences. Preferred organic ions include protonated tertiary amines and quaternary ammonium cations, including, in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

Also included in the combination of the invention are the isomeric forms and tautomers and the pharmaceutically-acceptable salts of the hexosamines. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include met allic ion salts and organic ion salts. More preferred met allic ion salts include, but are not limited to, appropriate alkali metal (group Ia) salts, alkaline earth met al (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The method and compositions of the present invention are useful for, but not limited to, the prevention, inhibition, and treatment of arthritis, and in particular, for rheumatoid arthritis and osteoarthritis. However, the present method and compositions are also useful for the prevention, inhibition and treatment of other articular joint damage or disease, including, but not limited to, spondyloarthopathies, gouty arthritis, systemic lupus erythematosus and juvenile arthritis. Also included as articular joint damage or disease are such conditions as tendinitis, bursitis, connective tissue injuries or disorders, and skin related conditions such as psoriasis, eczema, burns and dermatitis. In particular, the compositions of the invention are useful as anti-inflammatory agents, such as for the treatment of arthritis.

The terms “treating” or “to treat” means to alleviate symptoms, eliminate the causation either on a temporary or permanent basis, or to prevent or slow the appearance of symptoms. The term “treatment” includes alleviation, elimination of causation of or prevention of pain and/or inflammation associated with, but not limited to, any of the diseases or disorders described above. Besides being useful for human treatment, these combinations are also useful for treatment of mammals, including horses, dogs, cats, rats, mice, sheep, pigs, etc. It is believed that the subject hexosamines—glucosamine, in particular—might possess disease-modifying activity, which goes beyond treating signs and symptoms and includes activity that results in stabilization, partial or complete inhibition of further progression, or partial or total reversal of the disease.

The term “subject” for purposes of treatment includes any human or animal subject who is in need of the prevention of, or who has pain, inflammation and/or any one of the known inflammation-related disorders. The subject is typically a human subject, but companion animals, especially dogs, for example, are also viable subjects.

For methods of prevention, the subject is any human or animal subject, and preferably is a subject that is in need of prevention and/or treatment of pain, inflammation and/or an inflammation-related disorder. The subject may be a human subject who is at risk for pain and/or inflammation, or for obtaining an inflammation-related disorder, such as those described above. The subject may be at risk due to genetic predisposition, sedentary lifestyle, diet, exposure to disorder-causing agents, exposure to pathogenic agents, obesity, excessive joint use or damage (e.g., such as by certain athletes), and the like.

The pharmaceutical compositions may be administered enterally and parenterally. Oral (intra-gastric) is a preferred route of administration.

Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

In particular, the subject hexosamines can be administered orally, for example, as plants, plant parts, portions of plants that are enriched in hexosamines, as well as in the form of tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel hexosamines may be formulated with sweetening agents, for example glycerol, sorbitol or sucrose. Such formulations may also contain a demulcent, a preservative and flavoring and coloring agents.

The subject hexosamines can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed, including synthetic mon- or diglycerides. In addition, n-3 polyunsaturated fatty acids may find use in the preparation of injectables.

The subject hexosamines can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel hexosamines can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include capsules, tablets, food, and gelatin capsules, for example.

The present invention further comprises kits that are suitable for use in performing the methods of treatment, prevention or inhibition described above. In one embodiment, the kit contains a dosage form comprising glucosamine in one or more of the forms identified above, in a quantity sufficient to carry out the methods of the present invention. Preferably, the dosage form comprises a therapeutically effective amount of the hexosamine compound for the treatment, prevention, or inhibition of pain, inflammation or inflammation-associated disorder.

The following example describes preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. All amounts that are described in the examples in terms of percentage are to be understood to be percentage by weight, unless stated otherwise.

### EXAMPLE 1

This example illustrates that increasing levels of glucosamine introduced to a bovine nasal cartilage cell line concentration dependently inhibits IL-1 induced cartilage degradation.

Bovine nasal septum may be obtained fresh from a local slaughter house, or cleaned and prepared from Covance Research Products, Denver, Pa.

If the nasal septum tissue is fresh, it should be sanitized by wiping down with Betadine solution (Purdue Frederick Co., Norwalk, Conn.), and then placed in a disposable plastic container, such as a ZIPLOC® brand 76 fl. oz. container with lid, and rinsed quickly in an amount of 70% ethanol sufficient to almost cover the septum. The ethanol solution is then removed and replaced with phosphate buffered saline (PBS) solution with 2× antibiotic-antimycotic (available from GibcoBRL as catalog no.15246-062). After rinsing with the PBS solution, the septum tissue is then rinsed two times with Dulbecco's Modified Eagle's Medium (DMEM) solution with 2× antibiotic-antimycotic (available from GibcoBRL as catalog no.11995-040). The sanitized septum is then dissected by removing the skin, membranes and bone from the septum cartilage.

Cross-section slices (4 mm thick) are cut from either freshly dissected septum or prepared septum cartilage. This can be done with a meat slicer (such as General Slicing model #ES200, available from Hess Meat Machines, St. Louis, Mo.). Slices are placed in the DMEM solution described above in a disposable bottle.

Circular-shaped plugs of approximately 2 mm diameter are punched from the slices of the vascular section of the cartilage and placed in a tissue culture dish (such as 150×25 mm, Falcon cat. no. 3025) with assay medium composed of DMEM high-glucose with HEPES, without phenol red (available from GibcoBRL as catalog no.11360-070), plus 5 ml of 100× L-Glutamine 200 mM stock solution (available from GibcoBRL as cat. no. 25030-081). Just prior to use, sufficient ascorbic acid solution (available from Sigma, as cat. no. A-4034, filter sterilized) is added to bring the concentration of ascorbic acid to 50 μg/ml. The plugs are then incubated overnight at 37° C. in a 5% CO₂ atmosphere.

A stock solution of recombinant human IL-1 beta is prepared by resuspending 2 μg of IL-1 beta (available from R&D Systems as cat. no. 201-LB) in 1 ml sterile phosphate-buffered saline (available from GibcoBRL as cat. no.10010-023) containing 0.1% human serum albumin (available from Sigma as cat. no. A-8918). This stock solution is then diluted so that the final concentration of IL-1 beta in the medium is 3 ng/ml (i.e., 1.5 μl stock/ml of medium).

A stock solution of recombinant human oncostatin M is prepared by resuspending 10 μg lyophilized oncostatin M (available from R&D Systems, cat. no. 295-OM) in 100 ml sterile water. A sufficient amount of this stock solution is then added to bring the concentration of oncostatin M in the medium to 50 ng/ml. (i.e., 0.5 μl stock/ml of medium).

A stock solution of glucosamine is prepared by dissolving a sufficient amount of glucosamine (available, for example, as D(+)-glucosamine from Sigma, cat no. G-4875, or, alternatively, as glucosamine that is a product of the present method) in 100% dimethylsulfoxide (DMSO) (available from Sigma as cat. no. D-2650) to make a 2.5 mM solution.

In a 96-well tissue culture plate, 200 μl of medium containing IL-1 beta and oncostatin M is added to approximately one-half of the wells, and to the remaining wells add 200 μl of the same medium without the IL-1 beta or the oncostatin M for the unstimulated controls. Add one cartilage plug to each well.

Using the stock glucosamine solution, add a sufficient amount of the solution to pairs of wells with and without IL-1 beta and oncostatin M to provide a paired series of wells having glucosamine concentrations ranging from about 5 μM to about 500 μM and including positive control wells having zero glucosamine. DMSO is added to the positive control wells and to the unstimulated control wells to equal the amount of DMSO that was added with the glucosamine. The culture plate is then incubated in a 5% CO₂ atmosphere at 37° C.

Twice a week, supernatants are removed from each well and the solutions originally fed to the well are replenished with the same solutions made from fresh dilutions of the stored stock solutions. When the cartilage plugs in the unstimulated control wells have shrunk to a very small size, but before they disappear entirely, (usually about 11-15 days from the start of the incubation), the final supernatants are removed and the cartilage remains in the wells are covered with plate tape and the plates are frozen at −20° C. until ready to measure the amount of cartilage destruction by hydrolysis and hydroxyproline assay.

Hydrolysis and Hydroxyproline Assay

A stock papain solution in sterile water is prepared to contain: 25 mM sodium phosphate buffer, pH 6.5; 2 mM ethylenediaminetetracetic acid (EDTA) (at pH 8, available from GibcoBRL, cat. no.15575-038); 2 mM dithioerythritol (DTT) (FW=154.2, available from Sigma, cat. no. D-9779); and 20 Units/ml of papain (available from Sigma, as cat. no. P-4762).

Papain solution (200 μl) is added to each well of the tissue culture plate that contains the remains of a cartilage plug. All of the wells of the plate are then sealed with plate tape (available as Falcon 3073 pressure sensitive film), and the sealed plate is incubated at 60°-65° C. from 1-3 hours, or until all of the cartilage plugs are dissolved. After incubation, the plates can be stored at −20° C. until ready for hydrolysis and protein assay.

A sample of papain digestion solution from each well (25 μl) is transferred to a glass hydrolysis tube (6×50 mm, available as Corning #9820 culture tubes). The solutions in the tubes are then dried under vacuum and heat. By way of example, a Savant Speed Vac concentrator, Model no. ISS110-120, with model RH100-6 rotor will handle up to 100 tubes for this step.

Hydrolysis is performed by using a Pico-Tag Work Station (Millipore-Waters model #Pico Work S). Sample tubes containing dried residue from the papain hydrolysis are placed in a vacuum chamber vial (14 tubes fit into a Waters model WAT 007363 vial). Into each vial is added 500 μl of a solution of 6 N HCl containing 1% phenol. The vacuum vial is then loaded with sample tubes and sealed. Each vacuum vial is purged of air by applying vacuum for about 30 seconds and then refilling the vial with nitrogen. This is repeated for a total of four times. At the end of the final purge cycle, the valve is closed so that the vial remains under vacuum. The vial is placed in an oven at 150° C. for 1.5 hours. Immediately after the end of 1.5 hours, gaseous HCl is vented from the vial into a fume hood.

Each sample tube is removed from the vial, wiped off and dried for 20-30 minutes in the Savant system. When all sample tubes are dry, 250 μl of sterile milliQ water is added to each tube. After the tubes are allowed to sit overnight at 4° C., the contents are mixed by vortexing, and any remaining solids are formed into a pellet by centrifugation. About 200 μl of supernatant sample is removed from each tube and placed into a separate well in a 96 well tissue culture plate. The plate can be sealed with tape as described above until ready for the hydroxyproline assay.

The following stock solutions are prepared for the hydroxyproline assay:

Stock Buffer

23.052 g citric acid (Sigma cat. no. C-0759);

59.884 g NaOAc (sodium acetate trihydrate, mw=136.1, Sigma cat. no. S-9513);

36.093 g NaOAc (anhydrous sodium acetate, mw=82.03, Sigma cat. no. S-5889);

6.0 ml glacial acetic acid (17.4 M, Fisher cat. no. A38-212);

17 g NaOH (mw=40, Fisher cat no. S318-500); and

water (milliQ) sufficient to make up to 500 ml.

Assay Buffer

Prepare a mixture of n-propanol, milliQ water, and stock buffer in a 3:2:10 volume ratio (i.e., 30 ml, 20 ml, 100 ml).

Chloramine T Reagent

To prepare an amount sufficient for one 96-well culture plate, mix:

0.282 g chloramine-T (Sigma cat no. C-9887);

1.0 ml n-propanol (Sigma-Aldrich cat. no. 29,328-8);

1.0 ml milliQ water; and

8.0 ml stock buffer.

DMBA Reagent

To prepare an amount sufficient for one 96-well culture plate, mix:

2 g dimethylaminobenzaldehyde (DMBA, Sigma cat no. D-8904);

1.25 ml n-propanol; and

2.75 ml perchloric acid (Aldrich cat. no. 24,425-2

trans-4-hydroxy-L-proline

Prepare a stock solution of 3.2 mg/ml of trans-4-hydroxy-L-proline (Sigma cat no. H-6002) in sterile water and freeze in 1 ml aliquots.

To prepare a standard curve, add 24 μl of the trans-4-hydroxy-L-proline stock solution into 276 μl of water into well #1 of a 96-well culture plate. Add 150 μl of water to each of nine other wells. Serially dilute the trans-4-hydroxy-L-proline stock solution into the nine wells to provide trans-4-hydroxy-L-proline concentrations of 64, 32,16, 8, 4, 2,1, 0.5, and 0.25 μg/ml, and leave one well with water only to act as a 0 standard for the test. Pipet 15 μl of each sample into 45 =82 l of water into separate wells on an assay plate. These samples will provide the readings for construction of a standard curve.

Aliquot 50 μl of each hydrolyzed sample into 10 μl of water into separate wells in a 96-well assay plate.

Add 20 μl of assay buffer and 40 μl of chloramine T reagent to each well, and incubate at room temperature for 15 minutes. Add 80 μl of DMBA reagent to each well and mix. Incubate at 60° C. for 10 to 20 minutes. Remove from heat and allow to cool to room temperature for 5 minutes. Read the absorbance of each well at 560 nm against a 650 nm reference. Construct the standard curve and compute the protein concentrations for each test sample by comparing its absorbance against the standard curve.

The effect of glucosamine on the IL-1 beta-induced degradation of bovine nasal cartilage was measured by comparing the amount of hydroxyproline reported for a test sample (Cₜₑₛₜ) against the amount of hydroxyproline found for the cartilage standard having no IL-1 beta present in the medium (C_{std.}). The amount of cartilage computed as having been lost during the test due to IL-1 induced degradation (C_{std.}−Cₜₑₛₜ) could also be described as a fraction of the amount that was originally present. i.e., ((C_{std.}−Cₜₑₛₜ)/(C_{std.}). This value varied from zero-to-one, as the amount of cartilage that was degraded during the test increased.

The effect of glucosamine on the degradation was expressed in terms of the percent inhibition (% inhibition) that an amount of glucosamine provided against the IL-1 beta induced degradation. This value was calculated as:

% inhibition=(1−((C_{std.}−Cₜₑₛₜ)/(C_{std.}))×100.

Accordingly, in cases where glucosamine provided no inhibition, the value of % inhibition would be 0, whereas, if glucosamine provided total inhibition of the IL-1 beta induced degradation, the value of % inhibition would be 100.

The results from the test using glucosamine as a potential inhibitor of IL-1 beta induced cartilage degradation are shown in Table 1.

TABLE 1
Percent inhibition of IL-1 beta induced
cartilage degredation by glucosamine as a
function of glucosamine concentration.

| | GLUCOSAMINE | DEGREE OF INHIBITION OF |
|---|---|---|
| SAMPLE | CONCENTRATION | CARTILAGE DEGRADATION |
| NO. | (μM) | (%, mean and (Std. Dev.)) |
| 1 | 0 | 0 |
| 2 | 1.8125 | n/a |
| 3 | 3.625 | −1.9 (0.2) |
| 4 | 7.25 | 12.5 (17.6) |
| 5 | 14.5 | 51.5 (32.6) |
| 6 | 29 | 121.7 |
| 7 | 58 | 126.8 |

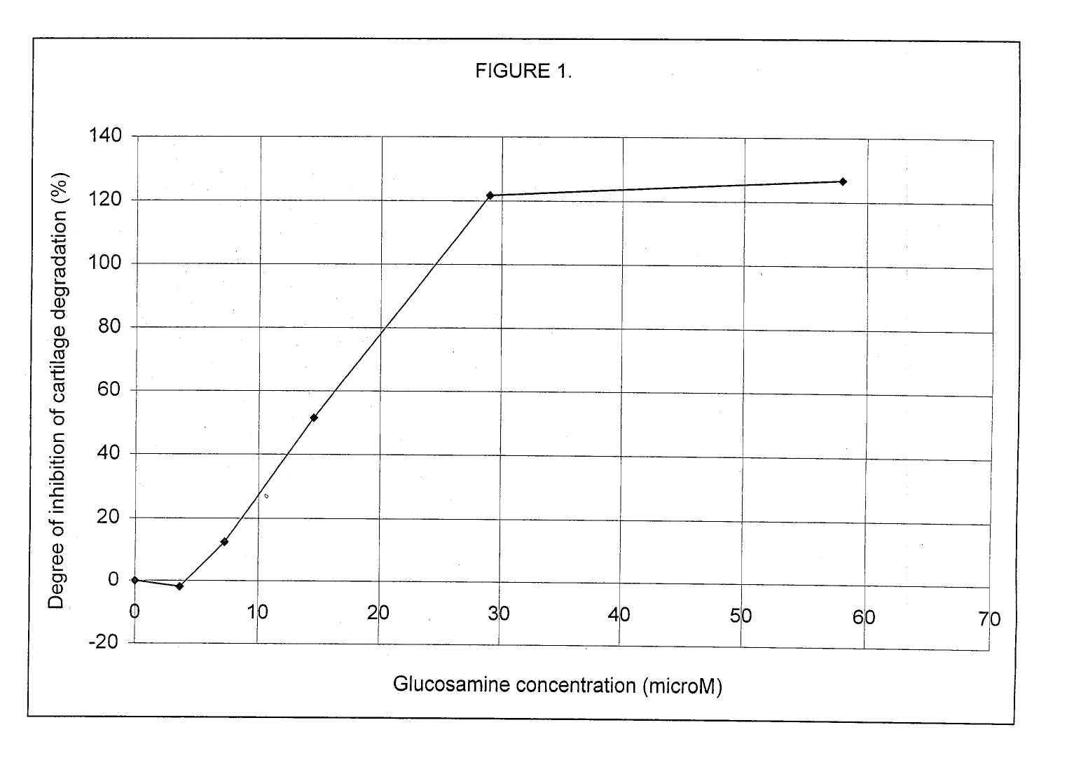

The values of the mean percent inhibition by glucosamine were plotted versus the glucosamine concentration in FIG. 1, which shows that glucosamine inhibits IL-1 beta induced degradation of bovine nasal cartilage in a dose-dependent manner between concentrations of about 3 μM, where substantially no inhibitory activity is seen, and about 30 μM, where substantially total inhibition of IL-1 beta induced degradation is seen.

It is believed that these data indicate that glucosamine is efficacious for inhibiting biological pathways of cartilage degradation that are typical in vivo for mammals.

All references cited in this specification, including without limitation all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained. As various changes could be made in the above methods and compositions without departing from the scope of the invention, it is intended that all matter contained in the above description and shown in the accompanying drawings shall be interpreted as illustrative and not in a limiting sense.

What is claimed is:

1. A method for producing a hexosamine comprising:
providing a cell comprising polynucleotide sequences which encode for enzymes required for a biosynthetic pathway capable of synthesizing the hexosamine, where at least one of the polynucleotide sequences comprises a recombinant polynucleotide.

2. The method according to claim 1, wherein the method further comprises culturing the cell under conditions that permit the cell to synthesize the hexosamine.

3. The method according to claim 1, wherein the cell is one that is genotypically negative for hexosamine synthesis.

4. The method according to claim 3, wherein the cell is a plant cell.

5. The method according to claim 1, wherein the recombinant polynucleotide encodes for glutamine:fructose-6-phosphate amidotransferase.

6. The method according to claim 1, wherein the recombinant polynucleotide encodes for glucosamine-6-phosphatase.

7. The method according to claim 5, wherein the recombinant polynucleotide encodes for glucosamine-6-phosphatase.

8. The method according to claim 5, wherein the recombinant polynucleotide encodes for phosphoglucomutase.

9. The method according to claim 8, wherein the recombinant polynucleotide encodes for glucosamine-1-phosphate acetyltransferase.

10. The method according to claim 9, wherein the recombinant polynucleotide encodes for acetyl-glucosamine-1-phosphate uridyltransferase.

11. The method according to claim 10, wherein the recombinant polynucleotide encodes for UDP-acetyl-glucosamine phrophophorylase.

12. The method according to claim 11, wherein the recombinant polynucleotide encodes for UCP-acetyl-glucosamine deacetylase.

13. The method according to claim 12, wherein the recombinant polynucleotide encodes for UCP-acetyl-glucosamine-C₄-epimerase.

14. The method according to claim 13, wherein the recombinant polynucleotide encodes for a phosphatase.

15. The method according to claim 1, wherein the hexosamine is selected from the group consisting of a glucosamine, a galactosamine, and a mannosamine.

16. The method according to claim 15, wherein the hexosamine comprises a glucosamine.

17. The method according to claim 16, wherein the glucosamine is selected from the group consisting of D-glucosamine, L-glucosamine, N-acetyl-glucosamine, UDP-N-acetyl-glucosamine, UDP-glucosamine, glucosamine-6-phosphate, and glucosamine-1-phosphate.

18. The method according to claim 17, wherein the hexosamine comprises D-glucosamine.

19. The method according to claim 15, wherein the hexosamine comprises a galactosamine

20. The method according to claim 19, wherein the galactosamine is selected from the group consisting of D-galactosamine, UDP-acetyl-galactosamine, and N-acetyl-galactosamine.

21. The method according to claim 20, wherein the galactosamine comprises D-galactosamine.

22. The method according to claim 1, wherein the hexosamine is selected from the group consisting of neuraminic acid, N-acetyl-neuramic acid, O-acetylneuraminic acid, N-glycolyineuraminic acid, muramic acid, and N-acetyl-muramic acid.

23. The method according to claim 15, wherein the hexosamine comprises a mannosamine.

24. The method according to claim 23, wherein the mannosamine is selected from the group consisting of D-mannosamine, and N-acetyl-D-mannosamine.

25. A method of producing a hexosamine comprising:
transforming a cell with at least one heterologous polynucleotide coding for a polypeptide in a biosynthetic pathway that is capable of producing a hexosamine; and
culturing the transformed cell under conditions that permit the cell to translate the polynucleotide into a polypeptide comprising an enzyme which is part of the biosynthetic pathway.

26. A method of producing a hexosamine by a living cell comprising:
constructing at least one heterologous vector encoding for at least one gene encoding a polypeptide that functions as an enzyme in a hexosamine biosynthetic pathway, wherein the gene is a polynucleotide operably linked to a regulatory promoter sequence;
transforming at least one cell with the heterologous vector;
culturing the cell under conditions which allow for expression of the polynucleotide into the polypeptide; and
permitting the cell to produce the hexosamine.

27. A method of producing hexosamines in transgenic plants comprising:
constructing at least one heterologous vector encoding for at least one gene in a hexosamine biosynthetic pathway and one gene for a plant selectable marker;
linking operably the hexosamine pathway gene and the plant selectable marker gene to at least one regulatory promoter that controls expression of said genes, wherein the expression of the genes is capable of producing a polypeptide for enzymatic hexosamine production and a polypeptide for the selectable marker;
transforming a plant cell with either one or both of the genes operably linked to the at least one regulatory promoter;
culturing the transformed plant cell under conditions that allow for the plant cell to regenerate into a plant; and
growing the transformed plant under conditions that allow for the plant to produce the hexosamine.

28. The method according to claim 27, comprising extracting the hexosamine from the plant.

29. The method according to claim 28, comprising purifying the hexosamine into a pharmaceutically useable form.

30. The method according to claim 27, wherein the plant cell is transformed by a method selected from the group consisting of:
Agrobacterium-mediated transformation, polyethylene glycol mediated uptake, microprojectile bombardment, viral-mediated transformation, and electroporation.

31. The method according to claim 27, wherein the promoter is selected from a group consisting of: a constitutive promoter, a viral promoter, an inducible promoter, a tissue-specific promoter, a plant promoter, a vegetable-specific promoter, a plant tuber-specific promoter, and a fruit-specific promoter.

32. The method according to claim 31, wherein the plant tissue-specific promoter is selected from the group consisting of: LeExp-1 promoter, patatin promoter, E8 promoter, MADS-box promoters, endo-β-1,4-glucanase promoter, expansin promoters, egase promoters, pectate lyase promoter, polygalacturonase promoters, and ethylene biosynthesis promoters.

33. The method according to claim 27, wherein the promoter is endogenous to the heterologous nucleic acid molecule.

34. The method according to claim 27, wherein the method comprises transforming a plant cell with a nucleic acid molecule encoding a glutamine:fructose-6-phosphate amidotransferase enzyme and at least one nucleic acid molecule encoding a polypeptide selected from the group consisting of: glucosamine-6-phosphatase, phosphoglucomutase, glucosamine-1-phosphate acetyltransferase, acetyl-glucosamine-1-phosphate uridyltransferase, UDP-acetyl-glucosamine pyrophosphorylase, UDP-acetyl-glucosamine-C₄-epimerase, and UDP-acetyl-glucosamine deacetylase.

35. The method according to claim 27, wherein at least one of the nucleic acid molecules encodes for a glutamine:fructose-6-phosphate amidotransferase enzyme.

36. The method according to claim 27, wherein at least one of the nucleic acid molecules encodes for a glucosamine-6-phosphatase enzyme.

37. The method according to claim 27, wherein the hexosamine biosynthetic pathway is derived from a eukaryotic hexosamine pathway.

38. The method according to claim 27, wherein the hexosamine biosynthetic pathway is derived from a prokaryotic hexosamine pathway.

39. The method according to claim 37, wherein the eukaryotic hexosamine pathway is selected from the group consisting of crustacean, insect, mollusk, fish, shark, mammal, algae, and fungal.

40. The method according to claim 38, wherein the prokaryotic hexosamine pathway is selected from the group consisting of bacteria.

41. A transgenic plant that is capable of producing a hexosamine, the plant comprising at least one heterologous vector encoding for at least one gene in a hexosamine biosynthetic pathway, wherein the gene is linked operably to at least one regulatory promoter that controls expression of the gene.

42. The plant according to claim 41, wherein the plant is transformed with a coding sequence of heterologous genes encoding for a glutamine:fructose-6-phosphate amidotransferase enzyme and a phosphotase enzyme, wherein the plant is capable of producing glucosamine.

43. The plant according to claim 42, wherein the plant is one having edible portions and wherein the plant is capable of producing glucosamine in the edible portions of the plant.

44. The plant according to claim 43, wherein the edible portion of the plant is selected from the group consisting of: edible fruit, leaves, roots, juices, stems, petioles, and seeds of the plant.

45. A recombinant expression system capable of expressing in host cells either one or both of:
a gene encoding for a glutamine:fructose-6-phosphate amidotransferase enzyme comprising a polynucleotide sequence that is substantially similar to SEQ ID NO: 1; and
a gene encoding for a phosphatase enzyme comprising a polynucleotide sequence that is substantially similar to SEQ ID NO: 13,
both genes being operably associated with a regulatory promoter sequence that controls expression of the genes, wherein the expression system is capable of producing glucosamine in the host cells.

46. The expression system according to claim 45, wherein the host cells comprise prokaryotic cells.

47. The expression system according to claim 46, wherein the host cells are bacterial cells.

48. The expression system according to claim 45, wherein the host cells are eukaryotic cells.

49. The expression system according to claim 48, wherein the host cells are selected from the group consisting of fungal cells, plant cells, and mammalian cells.

50. A method of treating a disease in a mammal comprising administering to the mammal by genetic therapy a recombinant expression vector capable of expressing in the mammal at least one least one gene in a hexosamine biosynthetic pathway, wherein the gene is linked operably to at least one regulatory promoter that controls expression of said the gene.

51. An edible material comprising a hexosamine produced from a recombinant source.

52. An edible material comprising plant material containing a hexosamine, wherein the plant is a transgenic plant that has been engineered to produce hexosamines.

53. The edible material according to claim 52, wherein the edible material is in a concentrated form from the transgenic plant.

54. The edible material according to claim 52, wherein the hexosamine is a glucosamine.

55. The edible material according to claim 52, wherein the hexosamine is a galactosamine.

56. The edible material according to claim 52, wherein the hexosamine is purified from the transgenic plant.

57. A method of producing glucosamine from a transgenic plant, the method comprising:
transforming a cell of the plant with a nucleic acid molecule having a heterologous nucleotide sequence substantially similar to SEQ ID NO: 1 encoding a glutamine:fructose-6-phosphate amidotransferase enzyme and a nucleic acid molecule having a heterologous nucleotide sequence substantially similar to SEQ ID NO: 13 encoding a phosphatase enzyme, both nucleic acids being operably linked to a regulatory promoter element;
culturing the transformed plant cell under conditions which allow the plant cell to regenerate into a plant;
growing the plant under conditions which allow the heterologous nucleic acid molecules to express the glutamine:fructose-6-phosphate amidotransferase and glucosamine-6-phosphatase enzymes;
allowing the heterologous enzyme glutamine:fructose-6-phosphate amidotransferase to convert fructose-6-phosphate into glucosamine-6-phosphate and the heterologous enzyme glucosamine-6-phosphatase to convert the glucosamine-6-phosphate into glucosamine;
extracting the glucosamine from the plant; and
purifying the extracted glucosamine into a pharmaceutically accepted form.

58. A transgenic host cell that is capable of producing a hexosamine, the cell comprising a cell having genes encoding each enzyme required for a biosynthetic pathway capable of synthesizing the hexosamine where at least one gene in the pathway is a heterologous gene.

59. The transgenic host cell according to claim 58, wherein the transgenic host cell is transformed with either one or both of a nucleic acid molecule encoding for a glutamine:fructose-6-phosphate amidotransferase enzyme having a nucleotide sequence substantially similar to SEQ ID NO: 1, and a nucleic acid molecule encoding for a phosphatase enzyme having a nucleotide sequence substantially similar to SEQ ID NO: 13, both genes being operably associated with a regulatory promoter sequence that controls expression of the genes, wherein the expression system is capable of producing glucosamine in the host cells.

60. A method for the prevention, treatment and/or inhibition of arthritis or articular joint damage or disease in a subject in need of such prevention, treatment, and/or inhibition, the method comprising administering to the subject a hexosamine-containing material which comprises polynucleotide sequences which encode for enzymes required for a biosynthetic pathway capable of synthesizing the hexosamine, where at least one of the polynucleotide sequences comprises a recombinant polynucleotide.


SEQUENCE LISTING (14 sequences)

SEQ ID NO: 1 - DNA, 2154 bp, Saccharomyces cerevisiae
atgtgtggtatctttggttactgcaattatctagtggaaagatccagaggagaaattatcgacaccttagtggatggtttacaaagattagaatatagaggctatgattccaccggtattgctatcgatggtgacgaagctgattctactttcatctataagcaaatcggtaaagtgagtgctttgaaagaggagattactaagcaaaatccgaacagagacgttacttttgtctctcattgtggtattgcgcatactagatgggctactcacggtcgaccagaacaagttaactgtcaccctcaaagatctgacccagaagaccaatttgtggtcgttcataatggtatcatcacaaattttagagaactgaagactcttttaattaacaaaggttataaattcgaaagtgataccgataccgagtgtattgctaaactatatttgcatttatacaatacaaatttacaaaatgggcatgacttagatttccacgaattaaccaagctagttcttttagaactagaaggttcatacgggttattatgtaaatcttgtcactatcctaatgaggttatcgccactagaaaagggtcccctttactgattggtgtcaaatctgaaaaaaaactaaaagtcgacttcgtggatgtggaatttcccgaagaaaacgctggtcaaccggaaattccattgaaatctaacaacaaatcatttggcttgggcccaaagaaagctcgtgaatttgaagctggttcccaaaatgccaatttactaccaattgccgccaatgaatttaacttgagacattctcaatccagggctttcctatcagaagatggatctccaacaccggtggaattttttgtttcttcggatgcggcatctgttgttaaacataccaagaaggtgctatttttagaagatgacgatttggctcatatttacgatggtgagttacatattcatagatctagaagagaagtaggcgcatcaatgacaaggtccattcaaactttagagatggagttagctcagatcatgaagggcccttacgaccattttatgcaaaaggaaatctatgagcaaccagaatctactttcaatactatgagaggtagaatcgactatgaaaataataaagtgatattgggtggtttaaaggcatggttaccagttgtcagaagagcacggagactgatcatgatcgcatgcggtacttcttatcattcatgtttggctactcgtgctatcttcgaagaattatcagatatcccagttagtgtggaattagcgtctgactttctggacagaaaatgccctgtcttcagagacgatgtatgcgtgtttgtttcacaaagtggtgaaactgcggataccatgctggctctaaattattgtttagaaagaggagccttaactgtcggaattgttaacagtgttggttcttctatctctcgtgtcacccactgtggtgttcatattaacgctggtcctgaaattggtgttgcctctacaaaagcttatacttcccagtatattgccttagtgatgtttgctctatcgctgtcagatgaccgtgtatcgaaaatagacagaagaattgaaatcattcaaggcttgaagttaatcccgggccaaattaagcaggtattaaagctggaaccaagaataaaaaagctctgtgcgactgaattaaaggatcaaaaatctctattgttattgggtagaggttaccaatttgctgctgctctggaaggtgctttgaagatcaaagaaatttcttatatgcattctgaaggtgttttggcaggtgagttgaagcacggtgtcttggccttggtggacgaaaacttgccaatcattgcttttggtaccagagactctctattccctaaagtagtttcctctattgagcaagttactgcaagaaagggccatccaattattatttgtaacgaaaatgatgaagtgtgggcgcaaaaatctaaatcaatcgacctgcaaaccttagaagttccacaaactgttgattgtttacaaggtctaattaatattattccattacaactaatgtcatattggttggctgttaataaagggattgatgttgattttccaagaaacttggctaaatctgttaccgtcgaataa

SEQ ID NO: 2 - protein, 717 aa, Saccharomyces cerevisiae
MCGIFGYCNYLVERSRGEIIDTLVDGLQRLEYRGYDSTGIAIDGDEADSTFIYKQIGKVSALKEEITKQNPNRDVTFVSHCGIAHTRWATHGRPEQVNCHPQRSDPEDQFVVVHNGIITNFRELKTLLINKGYKFESDTDTECIAKLYLHLYNTNLQNGHDLDFHELTKLVLLELEGSYGLLCKSCHYPNEVIATRKGSPLLIGVKSEKKLKVDFVDVEFPEENAGQPEIPLKSNNKSFGLGPKKAREFEAGSQNANLLPIAANEFNLRHSQSRAFLSEDGSPTPVEFFVSSDAASVVKHTKKVLFLEDDDLAHIYDGELHIHRSRREVGASMTRSIQTLEMELAQIMKGPYDHFMQKEIYEQPESTFNTMRGRIDYENNKVILGGLKAWLPVVRRARRLIMIACGTSYHSCLATRAIFEELSDIPVSVELASDFLDRKCPVFRDDVCVFVSQSGETADTMLALNYCLERGALTVGIVNSVGSSISRVTHCGVHINAGPEIGVASTKAYTSQYIALVMFALSLSDDRVSKIDRRIEIIQGLKLIPGQIKQVLKLEPRIKKLCATELKDQKSLLLLGRGYQFAAALEGALKIKEISYMHSEGVLAGELKHGVLALVDENLPIIAFGTRDSLFPKVVSSIEQVTARKGHPIIICNENDEVWAQKSKSIDLQTLEVPQTVDCLQGLINIIPLQLMSYWLAVNKGIDVDFPRNLAKSVTVE

SEQ ID NO: 3 - DNA, 480 bp, Saccharomyces cerevisiae
atgagcttacccgatggattttatataaggcgaatggaagagggggatttggaacaggtcactgagacgctaaaggttttgaccaccgtgggcactattacccccgaatccttcagcaaactcataaaatactggaatgaagccacagtatggaatgataacgaagataaaaaaataatgcaatataaccccatggtgattgtggacaagcgcaccgagacggttgccgctacggggaatatcatcatcgaaagaaagatcattcatgaactggggctatgtggccacatcgaggacattgcagtaaactccaagtatcagggccaaggtttgggcaagctcttgattgatcaattggtaactatcggctttgactacggttgttataagattattttagattgcgatgagaaaaatgtcaaattctatgaaaaatgtgggtttagcaacgcaggcgtggaaatgcaaattagaaaatag

SEQ ID NO: 4 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 5 - DNA, 1674 bp, Saccharomyces cerevisiae
atgaaggttgattacgagcaattgtgcaaactctacgatgacacgtgccgcacaaagaatgtgcagttcagttacggtacggccggattcagaacgctggccaagaatttggatacggtgatgttcagtactggtatactggcggttctcaggtcgctgaagcttcagggtcagtatgtgggggtgatgatcacggcgtcgcacaacccataccaggacaacggggtcaagatcgtggaaccagacggatcgatgcttttggccacatgggagccatatgccatgcagttggccaatgcggcctcttttgccactaattttgaagaatttcgtgttgagttggccaagctgattgaacacgaaaagattgatttgaatacaaccgtcgtgcctcacatcgtggttgggagagactctagggaaagtagtccatacttgctgcgctgcttgacttcctccatggccagcgtcttccacgcgcaagttttggacctaggctgtgtcactacgcctcaattgcattacattactgatttgtccaacaggcggaaactggaaggagacacagcgccagttgccacagaacaggactactattcgttctttataggagccttcaacgagctcttcgccacgtatcagctggagaagaggctgtctgtcccaaaattgttcatagacacagccaatggtatcggtggtccacagttgaaaaaactactggcctccgaagattgggacgtgccagcggagcaagttgaggtaatcaacgacaggtccgatgttccagaactgttgaattttgaatgcggtgcggattatgtgaagactaaccagagattacccaagggtctttctccatcctcgtttgattcgctatattgctcctttgatggtgacgcagacagggttgtgttctactatgtcgactcaggatcaaaatttcatttgttggatggtgacaaaatttccactttgtttgcaaagttcttgtctaaacaactagaattggcacacctagaacattctttgaagattggtgttgtgcaaactgcctatgcaaacggcagttccaccgcttacataaaaaatacgttgcactgtcccgtgtcttgcactaagacaggtgttaaacacttgcatcatgaagctgccactcagtacgatattggcatttatttcgaagcaaatggacatggtacgattatattcagcgaaaaatttcatcgaactatcaaatctgaattatccaagtccaagttaaatggtgatacgttagctttgagaactttgaagtgtttctctgaattgattaatcagaccgtgggagatgctatttcagacatgcttgctgtccttgctactttggcgattttgaaaatgtcgccaatggattgggatgaagagtatactgatttgcccaacaagctggttaagtgcatcgttcctgataggtcaattttccaaaccacggaccaggaaagaaaattgctcaatccagtggggttgcaagacaagatagatcttgtggtagccaagtatcccatgggaagaagctttgtcagagccagtggtacggaggatgcggtgagggtttatgcggaatgtaaggactcctctaagttaggtcaattttgtgacgaagtggtggagcacgttaaggcatctgcttga

SEQ ID NO: 6 - protein, 557 aa, Saccharomyces cerevisiae
MKVDYEQLCKLYDDTCRTKNVQFSYGTAGFRTLAKNLDTVMFSTGILAVLRSLKLQGQYVGVMITASHNPYQDNGVKIVEPDGSMLLATWEPYAMQLANAASFATNFEEFRVELAKLIEHEKIDLNTTVVPHIVVGRDSRESSPYLLRCLTSSMASVFHAQVLDLGCVTTPQLHYITDLSNRRKLEGDTAPVATEQDYYSFFIGAFNELFATYQLEKRLSVPKLFIDTANGIGGPQLKKLLASEDWDVPAEQVEVINDRSDVPELLNFECGADYVKTNQRLPKGLSPSSFDSLYCSFDGDADRVVFYYVDSGSKFHLLDGDKISTLFAKFLSKQLELAHLEHSLKIGVVQTAYANGSSTAYIKNTLHCPVSCTKTGVKHLHHEAATQYDIGIYFEANGHGTIIFSEKFHRTIKSELSKSKLNGDTLALRTLKCFSELINQTVGDAISDMLAVLATLAILKMSPMDWDEEYTDLPNKLVKCIVPDRSIFQTTDQERKLLNPVGLQDKIDLVVAKYPMGRSFVRASGTEDAVRVYAECKDSSKLGQFCDEVVEHVKASA

SEQ ID NO: 7 - DNA, 1434 bp, Saccharomyces cerevisiae
atgactgacacaaaacagctattcattgaagccggacaaagtcaacttttccacaattgggaaagcttgtctcgcaaagaccaagaagaattgctttcaaacctggagcaaatatcttccaagaggtcccctgcaaaactactggaagactgtcaaaatgctattaaattctcactagctaactcttctaaggatactggcgtcgaaatttcaccattgccccctacttcgtacgagtcgcttattggcaacagtaagaaagaaaatgaatactggcgtttaggccttgaagctattggcaagggtgaagtcgcagtgattttaatggctggcggacaaggtacgcggttaggatcctctcaaccaaagggctgttacgacattggattgccttctaagaaatctctttttcaaattcaagctgaaaagttgatcaggttgcaagatatggtaaaggacaaaaaggtagaaattccttggtatattatgacatcaggccccactagagcggctactgaggcatactttcaagaacacaattattttggcttgaataaagaacaaattacgttcttcaaccagggaaccctgcctgcctttgatttaaccgggaagcatttcctaatgaaagacccagtaaacctatctcaatcaccagatggaaatggtggactctaccgtgccatcaaggaaaacaagttgaacgaagactttgataggagaggaatcaagcatgtttacatgtactgtgtcgataatgtcctatctaaaatcgcagaccctgtatttattggttttgccatcaagcatggcttcgaactggccaccaaagccgttagaaagagagatgcgcatgaatcagttgggttaattgctactaaaaacgagaaaccatgtgtcatagaatattctgaaatttccaatgaattggctgaagcaaaggataaagatggcttattaaaactacgcgcaggcaacattgtaaatcattattacctagtggatttactaaaacgtgatttggatcagtggtgtgagaatatgccatatcacattgcgaagaagaaaattccagcttatgatagtgttaccggcaagtacactaagcctaccgaaccaaacggtataaaattagagcaattcatatttgatgtctttgacactgtaccactgaacaagtttgggtgcttagaagtagatagatgcaaagaattttcacctttaaaaaacggtcctggttctaagaacgataatcctgagaccagcagactagcatatttgaaactaggaacctcgtggttggaagatgcaggcgctattgtaaaagatggggtactagtcgaagtttccagcaaattgagttatgcaggtgaaaatctatcccagttcaaaggtaaagtctttgacagaagtggtatagtattagaaaaataa

SEQ ID NO: 8 - protein, 477 aa, Saccharomyces cerevisiae
MTDTKQLFIEAGQSQLFHNWESLSRKDQEELLSNLEQISSKRSPAKLLEDCQNAIKFSLANSSKDTGVEISPLPPTSYESLIGNSKKENEYWRLGLEAIGKGEVAVILMAGGQGTRLGSSQPKGCYDIGLPSKKSLFQIQAEKLIRLQDMVKDKKVEIPWYIMTSGPTRAATEAYFQEHNYFGLNKEQITFFNQGTLPAFDLTGKHFLMKDPVNLSQSPDGNGGLYRAIKENKLNEDFDRRGIKHVYMYCVDNVLSKIADPVFIGFAIKHGFELATKAVRKRDAHESVGLIATKNEKPCVIEYSEISNELAEAKDKDGLLKLRAGNIVNHYYLVDLLKRDLDQWCENMPYHIAKKKIPAYDSVTGKYTKPTEPNGIKLEQFIFDVFDTVPLNKFGCLEVDRCKEFSPLKNGPGSKNDNPETSRLAYLKLGTSWLEDAGAIVKDGVLVEVSSKLSYAGENLSQFKGKVFDRSGIVLEK

SEQ ID NO: 9 - DNA, 1011 bp, Yersinia enterocolitica
atgtctatattaattactggtggtgctggatatataggttctcatacagtgcttacattgttggaacaaggaaggaatgttgttgttcttgataatctaattaattcatcagcagagtccttagccagagtctcaaagatttgtggacgaaaacctaatttttatcatggcgatatacttgatagatcatgtcttaagcttattttttcaagtcataaaattgattcggttatccattttgcaggtttgaaatcagtaggtgagtcagttgaaaaacctattgagtattatcaaaacaatgtagttggttctattactttacttgaggaaatgtgtttagctaatgtcaaaaaattaatattcagctcttcagcaacagtatatggtgagcctgaattcgttccgttgactgaaaaagctagaattggcgggacgactaacccatatggcacttcaaaagtgatggtagagcaaatattaaaagatttctctttagctcatcccgactattcaataacagcgttgcgttattttaatccagtaggcgctcatccttctggtttaattggtgaggatcctaacggaaaacccaataatttattaccattcataacccaggttgccattggcaaattatctaaactattagtgtatggtaatgactacgatacaccagatgggtctggtattagagattatattcatgttatggatctcgctgaagggcaccttagtactttaattaacttgacctcaggatttcgtatatacaatttaggaaccggagttggctattctgttttgcatatgattaaggaatttgagcgtattacgggtaagaatattccatttgacattgttagccgtaggcctggtgatatagctgagtgttgggcaagccctgaactagcacatctagagttaggttggtatgcaaaaagaactttagttgatatgcttcaggacgcttggaaatggcaaaaaatgaatcctaatggttataactgttag

SEQ ID NO: 10 - protein, 336 aa, Yersinia enterocolitica
MSILITGGAGYIGSHTVLTLLEQGRNVVVLDNLINSSAESLARVSKICGRKPNFYHGDILDRSCLKLIFSSHKIDSVIHFAGLKSVGESVEKPIEYYQNNVVGSITLLEEMCLANVKKLIFSSSATVYGEPEFVPLTEKARIGGTTNPYGTSKVMVEQILKDFSLAHPDYSITALRYFNPVGAHPSGLIGEDPNGKPNNLLPFITQVAIGKLSKLLVYGNDYDTPDGSGIRDYIHVMDLAEGHLSTLINLTSGFRIYNLGTGVGYSVLHMIKEFERITGKNIPFDIVSRRPGDIAECWASPELAHLELGWYAKRTLVDMLQDAWKWQKMNPNGYNC

SEQ ID NO: 11 - DNA, 1170 bp, Escherichia coli
gtgagcgcaaaggcgctcgccgcttattcgaagagaatcgatgtgaaagtactgactgtatttggtacgcgcccggaagccatcaagatggcgccgttggtgcatgcgttggcaaaagatcctttttttgaggctaaagtttgcgtcactgcgcagcatcgggagatgctcgatcaggtgctgaaactcttttccattgtacctgactacgatctcaacataatgcagccaggacagggcctgacagagataacctgtcggattctggaagggctaaaacctattcttgccgagttcaaaccagacgtcgtgctggttcacggcgatacgacgacgacgctggcaaccagcctggcggcgttttatcagcgtattcctgttggtcacgttgaggctggtctgcgcacgggcgatctctattcgccgtggccggaagaggctaaccgtacattgaccgggcatctggcgatgtatcacttctctccaaccgaaacttcccggcaaaacttgctgcgtgaaaacgttgcggatagccgaatcttcattaccggtaatacagtcattgatgcactgttatgggtgcgtgaccaggtgatgagcagcgacaagctgtcagaactggcggcaaattacccgtttatcgaccccgataaaaagatgattctggtgaccggtcacaggcgtgagagtttcggtcgtggctttgaagaaatctgccacgcgctggcagacatcgccaccacgcaccaggacatccagattgtctatccggtgcatctcaacccgaacgtcagagaaccggtcaatcgcattctggggcatgtgaaaaatgtcattctgatcgatccccaggagtatttaccgtttgtctggctgatgaaccacgcctggctgattttgaccgactcaggcggcattcaggaagaagcgccttcgctggggaaacctgtgctggtgatgcgcgataccactgagcgtccggaagcggtgacggcgggtacggtgcgtctggtaggcacggataagcagcgaattgtcgaggaagtgacgcgtcttttaaaagacgaaaacgaatatcaagctatgagccgcgcccataacccgtatggtgatggtcaggcatgctctcgcattctggaagcgttaaaaaataatcggatatcactatga

SEQ ID NO: 12 - protein, 389 aa, Escherichia coli
MSAKALAAYSKRIDVKVLTVFGTRPEAIKMAPLVHALAKDPFFEAKVCVTAQHREMLDQVLKLFSIVPDYDLNIMQPGQGLTEITCRILEGLKPILAEFKPDVVLVHGDTTTTLATSLAAFYQRIPVGHVEAGLRTGDLYSPWPEEANRTLTGHLAMYHFSPTETSRQNLLRENVADSRIFITGNTVIDALLWVRDQVMSSDKLSELAANYPFIDPDKKMILVTGHRRESFGRGFEEICHALADIATTHQDIQIVYPVHLNPNVREPVNRILGHVKNVILIDPQEYLPFVWLMNHAWLILTDSGGIQEEAPSLGKPVLVMRDTTERPEAVTAGTVRLVGTDKQRIVEEVTRLLKDENEYQAMSRAHNPYGDGQACSRILEALKNNRISL

SEQ ID NO: 13 - DNA, 1074 bp, Homo sapiens
atggaggaaggaatgaatgttctccatgactttgggatccagtcaacacattacctccaggtgaattaccaagactcccaggactggttcatcttggtgtccgtgatcgcagacctcaggaatgccttctacgtcctcttccccatctggttccatcttcaggaagctgtgggcattaaactcctttgggtagctgtgattggagactggctcaacctcgtctttaagtggattctctttggacagcgtccatactggtgggttttggatactgactactacagcaacacttccgtgcccctgataaagcagttccctgtaacctgtgagactggaccagggagcccctctggccatgccatgggcacagcaggtgtatactacgtgatggtcacatctactctttccatctttcagggaaagataaagccgacctacagatttcggtgcttgaatgtcattttgtggttgggattctgggctgtgcagctgaatgtctgtctgtcacgaatctaccttgctgctcattttcctcatcaagttgttgctggagtcctgtcaggcattgctgttacagaaactttcagccacatccacagcatctataatgccagcctcaagaaatattttctcattaccttcttcctgttcagcttcgccatcggattttatctgctgctcaagggactgggtgtagacctcctgtggactctggagaaagcccagaggtggtgcgagcagccagaatgggtccacattgacaccacaccctttgccagcctcctcaagaacctgggcacgctctttggcctggggctggctctcaactccagcatgtacagggagagctgcaaggggaaactcagcaagtggctcccattccgcctcagctctattgtagcctccctcgtcctcctgcacgtctttgactccttgaaacccccatcccaagtcgagctggtcttctacgtcttgtccttctgcaagagtgcggtagtgcccctggcatccgtcagtgtcatcccctactgcctcgcccaggtcctgggccagccgcacaagaagtcgttgtaa

SEQ ID NO: 14 - protein, 357 aa, Homo sapiens
MEEGMNVLHDFGIQSTHYLQVNYQDSQDWFILVSVIADLRNAFYVLFPIWFHLQEAVGIKLLWVAVIGDWLNLVFKWILFGQRPYWWVLDTDYYSNTSVPLIKQFPVTCETGPGSPSGHAMGTAGVYYVMVTSTLSIFQGKIKPTYRFRCLNVILWLGFWAVQLNVCLSRIYLAAHFPHQVVAGVLSGIAVTETFSHIHSIYNASLKKYFLITFFLFSFAIGFYLLLKGLGVDLLWTLEKAQRWCEQPEWVHIDTTPFASLLKNLGTLFGLGLALNSSMYRESCKGKLSKWLPFRLSSIVASLVLLHVFDSLKPPSQVELVFYVLSFCKSAVVPLASVSVIPYCLAQVLGQPHKKSL